(19) United States
(12) Patent Application Publication
(10) Pub. No.: US 2022/0354943 A1
(43) Pub. Date: Nov. 10, 2022

(54) Therapeutic Agent Effectiveness and its Route of Adminstration

(71) Applicant: Altimmune, Inc, Gaithersburg, MD (US)

(72) Inventors: Vyjayanthi Krishnan, Gaithersburg, MD (US); M. Scot Roberts, Gaithersburg, MD (US)

(73) Assignee: Altimmune, Inc, Gaithersburg, MD (US)

(21) Appl. No.: 17/634,990

(22) PCT Filed: Aug. 13, 2020
(86) PCT No.: PCT/US20/46175

§ 371 Date: Feb. 13, 2022

Related U.S. Application Data
(60) Provisional application No. 62/886,234, filed on Aug. 13, 2019.

(51) Int. Cl. A61K 39/145; A61K 9/00; A61K 39/215; A61K 39/125; A61K 39/165; A61K 39/07; A61K 39/04; A61K 39/085; A61K 39/09; A61K 39/104; A61K 39/108; A61K 39/102; A61K 39/02; A61K 39/00; A61K 39/12; A61P 31/16; A61P 31/14

(52) U.S. Cl. CPC A61K 39/145; A61K 9/0043; A61K 39/215; A61K 39/125; A61K 39/165; A61K 39/07; A61K 39/04; A61K 39/085; A61K 39/092; A61K 39/104; A61K 39/0266; A61K 39/102; A61K 39/0241; A61K 39/0002; A61K 39/12; A61P 31/16; A61P 31/14; A61K 2039/545; A61K 2039/543

(57) ABSTRACT

Disclosed herein are methods for generating a protective immunogenic response via intranasal administration of an immunogenic composition (e.g., vaccine)/therapeutic immunogenic composition in a mammalian subject. Certain dosing positions of the subject during the administration of immunogenic agents, such that nostrils are tilted upwards, while in a modified sitting, reclining and/or supine posture, is surprisingly correlated with the generation of a strong immunogenic response in both humans and animals.

### RELATED APPLICATIONS

This application claims priority to U.S. Ser. No. 62/886,234 filed on 13 Aug. 2019, which is incorporated herein in its entirety.

### STATEMENT REGARDING FEDERALLY SPONSORED RESEARCH OR DEVELOPMENT

This invention was made with government support under contract HHSO100201600008C awarded by United States Government Biomedical Advanced Research and Development Authority (BARDA). The government has certain rights in the invention.

### FIELD OF THE DISCLOSURE

This application pertains generally to positional administration of an intranasal immunogenic composition (e.g., vaccine) dose at a therapeutic dose.

### BACKGROUND INFORMATION

Anthrax is an acute and severe disease caused by the toxin-producing, rod-shaped, Gram positive, aerobic bacterium Bacillus anthracis. Anthrax infection is mediated through environmentally stable spores that germinate after inoculation, ingestion, or inhalation with disease manifestations dependent on the route of infection (cutaneous, gastrointestinal, or inhalation anthrax). Case fatality rates over 90% are reported for the inhaled form (Wright J G, Quinn C P, Shadomy S, et al. Use of anthrax immunogenic composition (e.g., vaccine) in the United States: recommendations of the Advisory Committee on Immunization Practices (ACIP), 2009. MMWR Recomm Rep. 2010; 59(RR-6): 1-30).

B anthracis produces lethal toxins consisting of 3 proteins known as protective antigen (PA), lethal factor (LF), and edema factor (EF). PA binds to host cells and combines with LF or EF to form lethal toxin (LT) and edema toxin respectively (ET) [Wright 2010]. PA is a component of both toxins and antibody against PA is critically important to protection against anthrax disease.

B anthracis occurs naturally in the soil and causes animal infections, especially in large herbivores. Natural human infection usually occurs after contact with infected animals or animal hides but is very rare in the United States. Anthrax is considered one of the most likely agents to be used in a bioterrorism attack because the spores are very easy to grow in the laboratory, easy to disperse, and stable for long periods in the environment (Anthrax: The threat. Centers for Disease Control and Prevention Web site; September 2017). In 2001, 5 of the 22 people infected with anthrax, delivered via the US mail system in an act of bioterrorism, died.

The only immunogenic composition (e.g., vaccine) currently licensed for prevention of anthrax in the US is BioThrax (anthrax immunogenic composition (e.g., vaccine) adsorbed, AVA), manufactured by Emergent BioSolutions (Lansing, Mich.). This immunogenic composition (e.g., vaccine) is a sterile product made from cell-free filtrates of cultures of a strain of B anthracis not capable of causing disease. The schedule for pre-exposure prophylaxis is 3 doses administered intramuscularly at 0, 1, and 6 months. Booster doses at 12 months, 18 months, and annually thereafter are recommended for ongoing protection. The schedule for postexposure prophylaxis of disease is 3 doses administered subcutaneously at 0, 2, and 4 weeks after exposure in combination with antimicrobial therapy. These immunization schedules are inconvenient, time consuming, and associated with significant reactogenicity. The 2016 Public Health Emergency Medical Countermeasures Enterprise (PHEMCE) Strategy and Implementation Plan has determined that the approach for medical countermeasures includes the development of second-generation immunogenic composition (e.g., vaccine) against B anthracis (U.S. Department of Health and Human Services. 2016 Public health emergency medical countermeasures enterprise (PHEMCE) strategy and implementation plan. Rockville, Md.: U.S. Department of Health and Human Services; 2016).

Military personnel, other first responders, veterinarians, and other animal workers are vaccinated prior to exposure using the general use or pre-exposure schedule. In the event of bioweapon or other exposure, the US Centers for Disease Control and Prevention (CDC) recommend that unvaccinated individuals are given antibiotics such as ciprofloxacin or doxycycline to presumptively treat vegetative forms of B anthracis and are vaccinated concurrently to achieve protective levels of antitoxin antibody (Bell D M, Kozarsky P E, Stephens D S. Clinical issues in the prophylaxis, diagnosis, and treatment of anthrax. Emerg Infect Dis. 2002; 8:222-5; Hendricks K A, Wright M E, et al. Centers for disease control and prevention expert panel meetings on prevention and treatment of anthrax in adults. Emerg Infect Dis. 2014; 20). The antibiotics are discontinued once sufficient time has passed to achieve protective titer. Clearly, in the event of a public health emergency, the CDC recommendation requiring multiple doses of a painful, reactogenic immunogenic composition (e.g., vaccine) and a 60-day course of antibiotics presents significant logistical and public communication challenges. Second-generation immunogenic composition (e.g., vaccine) should have improved tolerability, more rapid onset of protection, and a simpler administration schedule to lessen burden on health care resources, which are likely to be in high demand after in the event of a large population exposure to a biologic toxin.

In one embodiment, this disclosure provides an exemplary second-generation biologic toxin immunogenic composition (e.g., vaccine) developed by the Applicant as NasoShield (previously known as AdVAV), a viral vectored anthrax immunogenic composition (e.g., vaccine) configured for intranasal administration and designed to provide rapid, stable protection after a single intranasal administration. In another embodiments, this disclosure provides an exemplary biologic influenza immunogenic composition (e.g., immunogenic composition (e.g., vaccine)) referred to herein as NasoVAX, and a new method for administering the same. The elimination of a booster immunogenic composition (e.g., vaccine) requirement and the simple intranasal administration of the immunogenic composition (e.g., vaccine) was a significant improvement, however the effectiveness varied. A need arose to determine why the effectiveness of an immunogenic composition (e.g., vaccine) can exhibit such variability and to provide an immunogenic composition (e.g., immunogenic composition (e.g., vaccine)) that protects against airborne infectious agents and biotoxins such as inhalation anthrax that will induce a protective immune response following administration. Applicants herein provide a method of administering such an immunogenic composition or formulation (e.g., immunogenic composition (e.g., vaccine)) wherein the effectiveness thereof immunogenic composition (e.g., vaccine) is significantly improved.

### SUMMARY OF THE DISCLOSURE

Herein some embodiments provided include compositions and methods for inducing an immune response, such as a protective immune response, against a pathogen via intranasal administration. In some embodiments, the pathogen is a respiratory pathogen.

In certain embodiments are provided methods for transmucosal administration of a therapeutic dose of an immunogenic composition (e.g., vaccine) comprising a respiratory antigen to a mammalian subject, wherein the method comprises administering intranasally to the mammalian subject an effective amount of the immunogenic composition (e.g., vaccine), wherein the subject during administration is in a sitting, reclining, supine, or other position wherein nostrils of the mammalian subject are tilted upward whereby the therapeutic dose administered transmucosally induces a protective immune response. In some embodiments, the mammalian subject is a human.

In certain embodiments are provided methods for transmucosal administration of a therapeutic dose of a non-replicating viral vectored immunogenic composition (e.g., vaccine) to a mammalian subject, wherein the method comprises administering intranasally to the mammalian subject the immunogenic composition (e.g., vaccine) comprising an effective amount of at least 10⁷ viral particle (vp) of replication deficient adenovirus vector that contains and expresses a heterologous antigen codon optimized for the mammalian subject, wherein the subject during administration is in a sitting, reclining, supine, or other position wherein nostrils of the mammalian subject are tilted upward whereby the therapeutic dose administered transmucosally induces a protective immune response. In some embodiments, the mammalian subject is a human. In certain embodiments, the heterologous antigen is from an influenza virus and the method is for treating and/or preventing infection by influenza virus and, in some preferred embodiments, prevents clinical worsening in an influenza patient. In some embodiments, the heterologous antigen is from a coronavirus virus and the method is for treating and/or preventing infection by a coronavirus such as SARS-CoV-2 (the causative agent of COVID-19) and, in some preferred embodiments, prevents clinical worsening in a COVID-19 patient.

In certain embodiments are provided methods for transmucosal administration of a therapeutic dose of a non-replicating viral vectored immunogenic composition (e.g., vaccine) to a mammalian subject, wherein the method comprises administering intranasally to the mammalian subject the immunogenic composition (e.g., vaccine) comprising an effective amount of at least 10⁷ viral particle (vp) of replication deficient adenovirus vector that does not contain and expresses a heterologous antigen, wherein the subject during administration is in a sitting, reclining, supine, or other position wherein nostrils of the mammalian subject are tilted upward whereby the therapeutic dose administered transmucosally induces a protective immune response. In embodiments, the induced protective immune response is against respiratory pathogens, including influenza, inhaled anthrax or coronavirus.

In certain embodiments, the present disclosure provides a method for inducing in a patient a protective immune response, comprising: intranasally administrating an effective amount of a therapeutic dose of an immunogenic composition to the patient, wherein the immunogenic composition comprises an antigen from a pathogen to a mammalian subject, wherein during the intranasal administration the patient is in a modified sitting, reclining, or supine, wherein nostrils of the mammalian subject are tilted upward; and upon administration, instructing the patient to maintain the modified sitting, reclining or supine position for 30 minutes after the intranasal administration, whereby the therapeutic dose administered intranasally induces a protective immune response in the patient.

In certain embodiments, the present disclosure provides a method for vaccinating a patient against viral infection, comprising: intranasally administrating an effective amount of a therapeutic dose of an immunogenic composition to the patient, wherein the immunogenic composition comprises an antigen from a pathogen to a mammalian subject, wherein during the intranasal administration the patient is in a modified sitting, reclining, or supine, wherein nostrils of the mammalian subject are tilted upward; and upon administration, instructing the patient to maintain the modified sitting, reclining or supine position for 5 minutes, 10 minutes, 15 minutes, 20 minutes, 25 minutes, or 30 minutes after the intranasal administration, whereby the therapeutic dose administered intranasally induces a protective immune response in the patient.

In certain embodiments , the present disclosure provides a method for treating viral infection in a patient, comprising: intranasally administrating an effective amount of a therapeutic dose of an immunogenic composition to the patient, wherein the immunogenic composition comprises an antigen from a pathogen to a mammalian subject, wherein during the intranasal administration the patient is in a modified sitting, reclining, or supine, wherein nostrils of the mammalian subject are tilted upward; and upon administration, instructing the patient to maintain the modified sitting, reclining or supine position for 5 minutes, 10 minutes, 15 minutes, 20 minutes, 25 minutes, or 30 minutes after the intranasal administration, whereby the therapeutic dose administered intranasally induces a protective immune response in the patient.

Other embodiments are also contemplated as will be understood by those of ordinary skill in the art from this disclosure.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows the schematic diagram of a representative adenoviral vector containing the anthrax protective antigen (PA) gene, wherein the numbers refer to base pair number in wild-type Ad5 sequence, GenBank ID AY339865.1.

FIG. 2 shows the lack of immunogenicity via a toxin neutralizing assay (TNA) in a Phase 1 NasoShield study wherein human subjects were administered NasoShield at four different viral particle (vp) concentrations as compared to BioThrax and placebo. See Example 2.

FIG. 3 shows the toxin neutralizing assay (TNA) results from a pre-clinical rabbit study to support the first in man clinical trial of Example 2.

FIG. 4 shows study design of a rabbit study to compare the positional effect (e.g. sternal or dorsal position) of the rabbit during administration of the immunogenic composition as to the immunogenicity of intranasal administration of NasoShield.

FIG. 5 shows TNA ED₅₀ and TNA NF₅₀ from vaccinated rabbits evaluated in a validated TNA assay at day 28 post vaccination. The sternal position for administration showed no immunogenicity in TNA assay.

FIG. 6 shows the positional effect on immunogenicity as measured by toxin neutralization assay (TNA), a functional assay that measures and quantifies the ability of serum to neutralize B. anthracis lethal factor (LT), when immunogenic composition is administered intranasally.

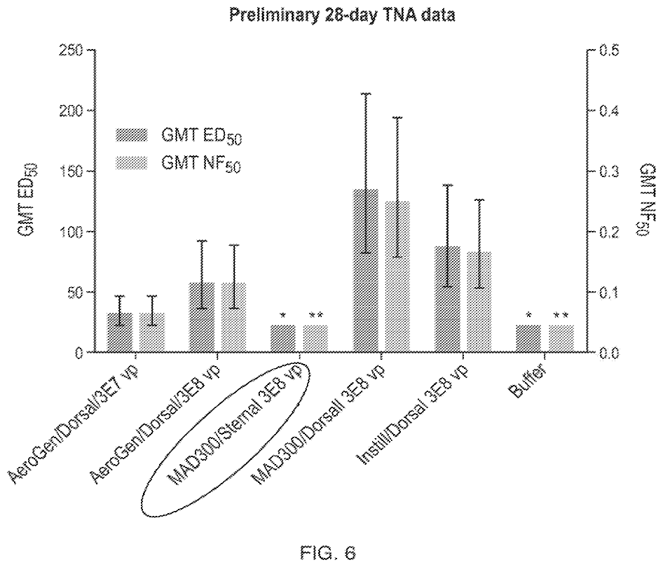

FIG. 7 shows the percent survival of rabbits administered NasoShield in a dorsal (e.g. standard rabbit position for intranasal administration) or sternal (clinical equivalent position) post Anthrax spore challenge. The animals administered NasoShield in a sternal position demonstrated 0% survival post Anthrax challenge as compared to a survival of 54% to 79% for animal administered the immunogenic composition in a dorsal position.

FIG. 8 shows TNA NF₅₀ results as compared to survival of rabbits in each of the different dosing groups.

FIG. 9 shows study design of a rabbit study to compare the positional effect (e.g. sternal or dorsal position) of the rabbit during administration of the immunogenic composition as to the immunogenicity of intranasal administration of NasoVAX.

FIG. 10 shows HAI assay Geometric Mean Titer from vaccinated rabbits evaluated in at days 0, 14 and 28 post-vaccination.

FIG. 11 shows HAI assay Geometric Mean Titer from vaccinated rabbits evaluated in at days 0 and 28 post-vaccination.

FIG. 12 shows HAI assay Geometric Mean Ratio (GMR) from vaccinated rabbits evaluated in at days 14 and 28 post-vaccination.

FIG. 13 shows Average HAI assay Score from vaccinated rabbits evaluated from days 0 to 28 post-vaccination.

### DETAILED DESCRIPTION

Introduction

This disclosure provides compositions and methods for generating a strong immunogenic response via intranasal administration of an immunogenic composition (e.g., vaccine)/therapeutic composition to a mammalian subject. In some embodiments, certain positions of the subject during the administration of immunogenic agents, such that nostrils are tilted upwards, while in a sitting, reclining and/or supine posture, is correlated with the generation of a strong immunogenic response in both humans and animals. The position of administration (e.g., the position of the head/nostrils during intranasal administration) was surprisingly identified as a critical parameter for generating an immunogenic response for intranasally administered immunogenic compositions (e.g., vaccines) and potentially other therapeutics. See Examples 3-4. Applicants postulated that tilting the head (nostrils angled upward) may allow for the therapeutic agent/immunogenic composition (e.g., vaccine) to access and/or localize in specific areas of the nasal passages, sinuses and adjacent tissues/organs, which may be critical for eliciting an immune response via transmucosal administration. The areas accessed by intranasal administration in sternal (in animals) and sitting posture with nostrils facing downward (in humans) may be different and/or sub-optimal for generating a strong immune response.

In certain embodiments provided herein are methods for transmucosal administration to a mammalian subject of a therapeutic dose of an immunogenic composition (e.g., vaccine) comprising an antigen from a pathogen and/or one or more heterologous antigens, wherein the method comprises administering intranasally to the mammalian subject an effective amount of the immunogenic composition (e.g., vaccine), wherein the subject during administration is in a supine, reclining, or other position wherein nostrils of the mammalian subject are tilted upward or above the head of a subject and whereby the therapeutic dose administered induces a protective immune response. In certain embodiments provided herein are methods for transmucosal administration to a mammalian subject of a therapeutic dose of a non-replicating viral vectored immunogenic composition to a mammalian subject, wherein the non-replicating viral vectored immunogenic composition comprises a replication deficient adenovirus vector, with or without containing and expressing a heterologous antigen codon optimized coding sequence for the mammalian subject, and wherein the method comprises administering intranasally to the mammalian subject an effective amount of the immunogenic composition (e.g., vaccine), wherein the subject during administration is in a supine, reclining, or other position wherein nostrils of the mammalian subject are tilted upward or above the head, and whereby the therapeutic dose administered induces a protective immune response. In certain embodiments, the protective immune response is against a respiratory pathogen. In certain embodiments, the protective immune response is an innate immune response that may be elicited within 24 hours or 1 to days, and can last up to 14 days, or longer. In certain embodiments, the mammalian subject is a human.

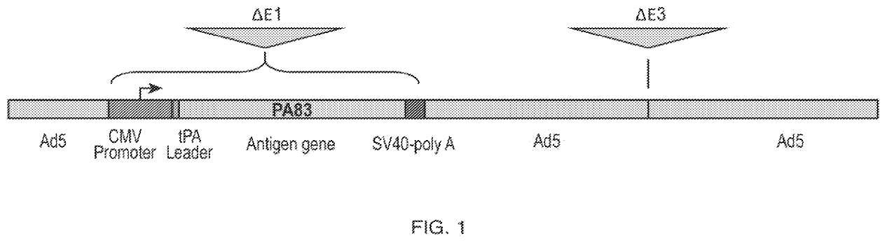

In one embodiment, this disclosure provides an adenoviral vector (e.g., Ad5-vectored), intranasal anthrax immunogenic composition (e.g., vaccine) produced in cell culture (NasoShield), see U.S. Pat. No. 9,968,667, the content of which is incorporated by reference in its entirety. Adenovirus is a naturally occurring respiratory virus that has been used frequently as a vector to introduce genetic material into cells. By incorporating the B. anthracis protective antigen (PA) gene into replication-deficient (RD) adenovirus (Ad-PA) and applying the Ad-PA into the nose (intranasal route of administration), the adenoviral vector can transduce the PA gene into cells of the nasal sub-mucosa, leading to transient expression of the encoded PA protein. NasoShield is typically delivered intranasally, but this disclosure demonstrates the immunogenic composition (e.g., vaccine) surprisingly induced a strong immunogenic response when administered such that the nostrils are tilted upwards or above the head (i.e., the modified sitting, dorsal or supine position). The NasoShield vector contains a genetic insert such as the transgene encoding the PA₈₃ (protective antigen—83 kDa form) gene from B anthracis (or any other gene of interest in trans). The recombinant Ad5 vector lacks the E1 region of the viral genome which renders the virus replication deficient (RD) and incapable of producing infectious virus particles upon entry into a host cell. An additional deletion of nucleotides in the E3 region of the vector removes genes that are involved in evading the host immune response and are dispensable for virus replication. An expression cassette consisting of a cytomegalovirus transcriptional enhancer/promoter to drive the expression of the PA₈₃ gene, (or other specific transgene), and a Simian Virus 40 polyadenylation signal has been inserted in place of the E1 gene sequences. FIG. 1 provides a schematic diagram of the RD-Ad5 vector and identifies those sequences from the parent adenovirus genome that are retained in the vector. See Example 1, 3 and 5.

As understood by one of skill in the art, and in some embodiments herein, the measurement of toxin neutralizing antibody (TNA) response can be used as a “surrogate of protection”. For example, in published studies a TNA NF₅₀ antibody (toxin neutralizing antibodies to PA) titer of 0.56 measured in post vaccination rabbit serum was associated with a 70% probability of surviving a lethal challenge with Bacillus anthracis spores (Ionin et al. Evaluation of Immunogenicity and Efficacy of Anthrax Vaccine Adsorbed for Postexposure Prophylaxis; Clin. and Vaccine Imunol.; July 2013: 20(7); p. 1016-1026). A “surrogate of protection” means an immune marker that can substitute for the clinical end point and thus, can be used to reliably predict immunogenic composition (e.g., vaccine) efficacy (Plotkin S.; Correlates of Protection Induced by Vaccination; Clin Immunogenic composition (e.g., vaccine) Immunol. 2010 July; 17(7): 1055-1065).

In another embodiment, this disclosure provides an adenoviral vector (e.g., Ad5-vectored), intranasal influenza (“flu”) immunogenic composition (e.g., vaccine) produced in cell culture (NasoVAX). Adenovirus is a naturally occurring respiratory virus that has been used frequently as a vector to introduce genetic material into cells. By incorporating the influenza hemaglutinnin (HA) gene into replication-deficient (RD) adenovirus (Ad-HA) and applying the Ad-HA into the nose (intranasal route of administration), the adenoviral vector can transduce the HA gene into cells of the nasal sub-mucosa, leading to transient expression of the encoded HA protein. In some embodiments, a replication-deficient adenoviral vector containing and expressing influenza virus hemagglutinin antigen codon optimized for the human subject (e.g., NasoVAX) can be prepared following the procedure detailed in Lui J. et al. (A protocol for rapid generation of recombinant adenoviruses using the AdEasy system; Nat. Protoc. (2007) 2(5):1236-47). NasoVAX, as used in the Examples herein, is an E1/E3-deleted, replication deficient (RD)-Ad5 vector that expresses the protein of interest (e.g., influenza HA type A or B) within respiratory epithelial cells. Thus, NasoVAX lacks the E1 region of the viral genome (nucleotides 343 to 3511), which renders the virus replication defective (RD) and incapable of producing infectious virus particles upon entry into a host cell. NasoVAX includes an additional deletion of nucleotides in the E3 region of the vector that removes genes that are involved in evading the host immune response and are dispensable for virus replication. NasoVAX also includes an expression cassette consisting of a cytomegalovirus transcriptional enhancer/promoter to drive the expression of the HA gene, a bioengineered HA gene, and a Simian Virus 40 polyadenylation signal has been inserted in place of the E1 gene sequences. FIG. 1 provides a schematic diagram of the RD-Ad5 vector and identifies those sequences from the parent adenovirus genome that are retained in the NasoVAX vector. In certain embodiments, the NasoVAX vector contains a genetic insert encoding the hemagglutinin (HA) surface protein antigen from an A/California/04/2009(H1N1)-like strain of influenza (AdcoCA09.HA). NasoVAX can be manufactured by propagation of this RD-Ad5 vector in replication-permissive PER.C6 cells, followed by purification of the virus from the infected cell harvest, and the final product can include excipients selected from Tris HCl (pH 7.4), histidine, sucrose, sodium chloride, magnesium chloride, polysorbate 80, ethylenediaminetetraacetic acid, and ethanol. NasoVAX can be provided in single-use glass vials each containing a nominal volume of 0.7 mL of a sterile, frozen suspension of immunogenic composition (e.g., vaccine), in some embodiments formulated as 2×10¹⁰ vp/mL in A195 buffer. In preferred embodiments, NasoVAX can be delivered intranasally, and this disclosure demonstrates the immunogenic composition (e.g., vaccine) surprisingly induced a strong immunogenic response when administered such that the nostrils are tilted upwards or above the head (i.e., modified sitting, supine or reclining (e.g., dorsal position)). See, e.g., Examples 4 and 6.

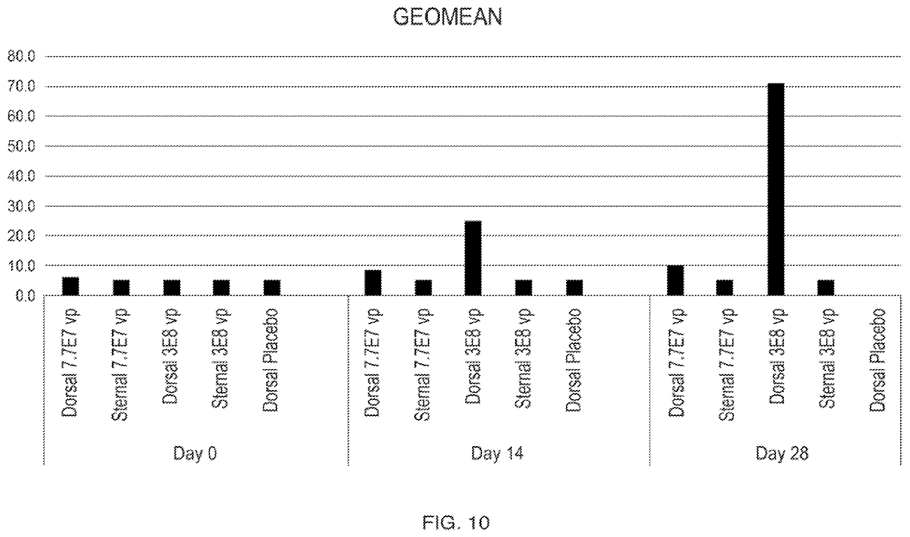

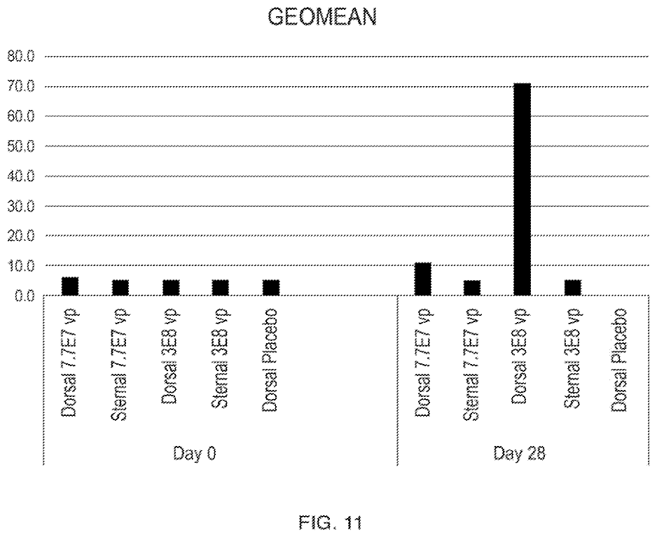

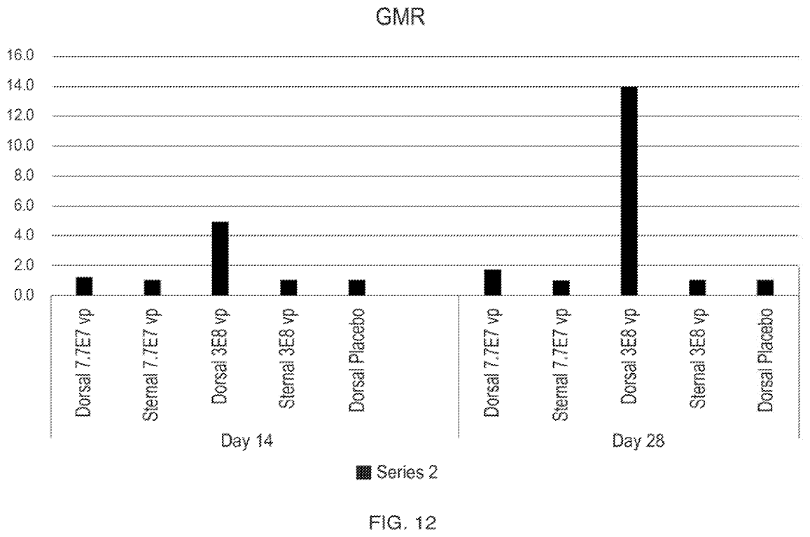

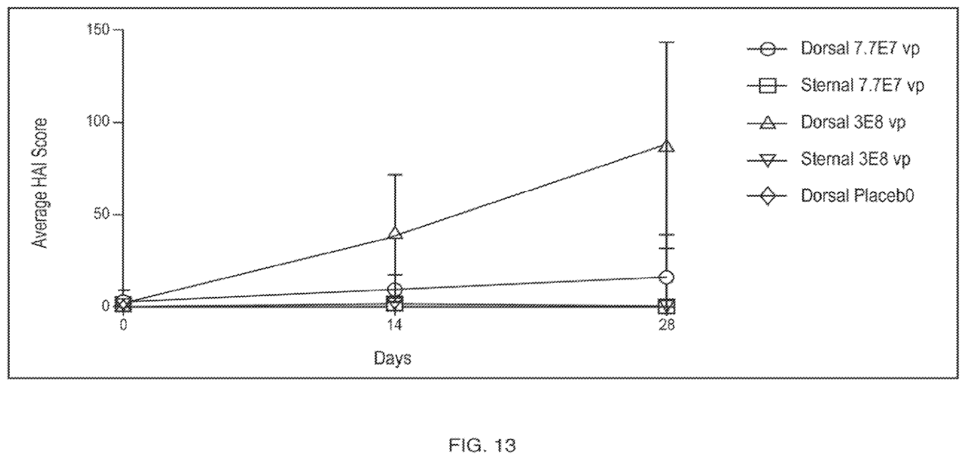

As understood by one of skill in the art, the measurement of hemagglutination inhibition assay (HAI) antibodies is used as a surrogate of protection wherein a HAI antibody titer of ≥40 measured in post vaccination serum demonstrates induced seroprotection by the administered influenza immunogenic composition (e.g., vaccine) (Trombetta C M. et al.; Overview of Serological Techniques for influenza immunogenic composition (e.g., vaccine) Evaluation: Past, Present and Future Immunogenic composition (e.g., vaccine) (Basel) (2014) December 2 (4): 707-734). As mentioned above, a “surrogate of protection” refers to an immune marker that can substitute for the clinical end point and thus, can be used to reliably predict immunogenic composition (e.g., vaccine) efficacy. In the case of influenza immunogenic composition (e.g., vaccine), antibodies measured in a HAI assay is a surrogate of protection, wherein the HAI assay is based on the ability of antibodies, if present in the serum, to prevent agglutination between erythrocytes and viral hemagglutinin. It is generally understood that subjects with a HAI assay antibody titer of ≥40 are protected from represented influenza A virus subtypes and influenza B virus. In other words, an HAI assay antibody titer of 40 (or greater) is generally considered as a protective threshold level, beyond which there is a 50% or greater reduction in the possibility of contracting an influenza infection. An HAI assay titer equal to or greater than 40 is used as an immunological correlate of protection and is regarded as the best currently available parameter for predicting protection from natural infection, according to FDA guidelines for pandemic influenza vaccines (Noah D. L., Hill H., Hines D., White E. L., Wolff M. C. Qualification of the hemagglutination inhibition assay in support of pandemic influenza vaccine licensure. Clin. Immunogenic composition (e.g., vaccine) Immunol. 2009; 16:558-566). In some embodiments, use of HAI assay to measure the immune response following administration of an immunogenic composition (e.g., vaccine, NasoVAX) is described in Example 4 and the results presented in FIGS. 10-13. Other embodiments of HAI assay and its use are also contemplated herein as would be understood by those of ordinary skill in the art.

In one embodiment, this disclosure provides an adenoviral vector (e.g., Ad5-vectored), intranasal COVID-19 immunogenic composition (e.g., vaccine) produced in cell culture. In certain embodiments, the heterologous antigen is a coronavirus virus antigen (e.g., spike protein, or fragment thereof) and the method is for treating and/or preventing infection by a coronavirus such as SARS-CoV-2 (the causative agent of COVID-19) and, in some preferred embodiments, prevents clinical worsening in a COVID-19 patient. By incorporating a SARS-Cov-2 antigen (e.g., spike protein) gene into replication-deficient (RD) adenovirus (AdCOVID) and applying the AdCOVID into the nose (intranasal route of administration), the adenoviral vector can transduce the protein antigen gene into cells of the nasal sub-mucosa, leading to transient expression of the encoded antigen protein or fragment thereof. In certain embodiments, the replication-deficient (RD) adenovirus does not incorporate a heterologous antigen (e.g., transgene from a different respiratory pathogen) (also referred to herein as “AdE”), but which when administered intranasally with the subject in a posture with the nostrils tilted up or above the head, induced a protective immune response against respiratory pathogens, including SARS-Cov-2. Replication deficient adenoviral vector binds to its receptor on cells of the nasal sub-mucosa eliciting an innate immune response, which may be protective against respiratory pathogens in humans. In embodiments, the protective immune response is an innate immune response that is induced within about 24 hours, or within 1-2 days, and may last at least about 14 days or at least about 21 days. This disclosure, at least in part, is directed to a disclosed immunogenic composition (e.g., vaccine) that surprisingly induced a strong immunogenic response when administered such that the nostrils are tilted upwards or above the head (i.e., the modified sitting, reclining (dorsal) or supine position). See Example 6.

Other embodiments are also contemplated as will be understood by those of ordinary skill in the art from this disclosure.

Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

The compositions, formulations and methods of the present invention may comprise, consist essentially of, or consist of the components and ingredients of the present invention as well as other ingredients described herein. As used herein, “consisting essentially of” means that the compositions, formulations and methods may include additional steps, components or ingredients, but only if the additional steps, components or ingredients do not materially alter the basic and novel characteristics of the claimed compositions, formulations and methods.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, an “adjuvant” refers to a separate/second substance that enhances the body's immune response to an antigen. In some embodiments, the present monovalent influenza pharmaceutical formulation is a non-adjuvanted immunogenic composition (e.g., vaccine).

By “administration” is meant introducing an immunogenic composition (e.g., vaccine) of the present disclosure into a subject; it may also refer to the act of providing a composition of the present disclosure to a subject (e.g., by prescribing).

The term “dosing position” as used herein refers to the position of the subject during intranasal administration of the present immunogenic compositions and, optionally, during the post-dose hold period. Those dosing positions include: a) a normal sitting position with the nostrils pointed down, the position may be maintained wherein the head of the subject is facing directly forward with eyes focused straight ahead on an object at eye level; or 2) a modified sitting, reclining, supine, or other position wherein nostrils of the mammalian subject are tilted upward. A modified sitting position means a dosing position wherein the subject is sitting with nostrils elevated above the head. This is accomplished, for example, with the head tilted back and chin lifted as far as comfortably possible with eyes focused on the ceiling (or sky if outdoors). A supine position means a position positions wherein the subject is laying flat on their back (without a pillow) with head facing forward and eyes focused straight ahead on the ceiling (or sky if outside). In certain embodiments, the present immunogenic compositions are administered via an intranasal device (e.g. atomizer) angled slightly inwards toward septum of the nose with the device (e.g. syringe barrel) in place with the facial plane.

The term “therapeutically effective amount” or “effective amount” as used herein refers to that amount of the compound being administered which will induce an innate, mucosal, humoral and/or cell mediated immune response, wherein the immune response may be understood by those of ordinary skill in the art to be immunogenic, therapeutic and/or protective (e.g., as a vaccine). The term can also refer to an amount of the present compositions, or route of administering the same such as disclosed herein, that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in a mammal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof (e.g., as a vaccine). The term “effective amount” refers to that amount of the compound being administered which will produce a reaction that is distinct from a reaction that would occur in the absence of the compound.

The term “therapeutic dose” as used herein refers to an amount of the present immunogenic composition, configured and/or formulated for intranasal administration, that provides a therapeutically effective amount. In certain embodiments, the therapeutic dose is present in a divided dose meaning a first portion is administered into one nostril and a second portion is administered into the second nostril and in combination provide the therapeutic dose.

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein, a “pharmaceutically acceptable carrier” refers to a carrier or diluent that does not cause significant irritation to the human subject and does not abrogate the biological activity and properties of the administered immunogenic composition (e.g., vaccine).

As used here, the term “seroconversion” is defined as a 4-fold or greater increase in serum neutralization antibody titers after vaccination (e.g., administration of a present immunogenic composition). In the context of influenza, the term “seroconversion” is a rate defined as the percentage of individuals vaccinated (administered a present immunogenic composition (e.g., vaccine) formulation) who have at least a 4-fold increase in serum haemagglutinin inhibition (HI) titers after vaccination. As used herein “conversion factor” is defined as the fold increase in serum HI geometric mean titers (GMTs) after vaccination.

As used herein, the term “seroprotected” means a subject post vaccination that is protected from infection via generation of serum neutralization antibodies. In a population, this is referred to as a percentage (%) of seroprotected individuals (e.g., 50%). In the context of influenza, the term “seroprotection” refers to an HA1 antibody titer of 40 or greater measured in serum from a human subject post-vaccination. The term “protection rate” as used herein in the context of influenza is defined as the percentage of individuals vaccinated with a serum HAI titer equal to or greater than 1:40 after vaccination and is normally accepted as indicating protection. In embodiments, the present immunogenic compositions and methods of use provide seroprotection to the mammalian subject, such as a human subject, against SARS-CoV-2 infection. In certain embodiments, the present immunogenic compositions and methods of use provide seroprotection to the mammalian subject, such as a human subject, against B. anthracis infection.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

As used herein, an “immunogenic composition” refers to a composition that when administered induces an immune response (e.g., innate, humoral, mucosal and/or cell mediated). In the instance of a humoral, mucosal and/or cell mediated immune response, the immunogenic composition induces an immune response against the antigen, and by extension the pathogen it was derived from, administered to the host. In the instance of an innate immune response, as elicited by an intranasal administered adenoviral vector, the immunogenic composition induces an immune response against respiratory pathogens, which may be protective for a period of about 24 hours to at least 14 days. As used herein “vaccine” refers to an immunogenic composition that when administered induces a protective immune response against an infectious agent. A “protective immune response” is one that protects the mammalian subject (e.g., patient) from infection by, and/or alleviates the symptoms of infection by, a pathogen expressing or otherwise susceptible to an immune response against at least one component in the immunogenic composition, typically at least one antigen (e.g., heterologous antigen) thereof. In certain embodiments, as used herein protective immune response refers to a pan-anti-viral innate immune response that protects the mammalian subject (e.g., patient) from infection by, and/or alleviates the symptoms of infection. In certain embodiments, an immunogenic composition (e.g., vaccine) can comprise one or more viral vector(s), with or without, containing and/or expressing a heterologous antigen, along with other components of an immunogenic composition (e.g., vaccine) suitable for administration to a mammalian host, including for example one or more adjuvants, slow release compounds, solvents, buffers, etc. In certain embodiments, an immunogenic composition and/or vaccine can comprise a protein and/or carbohydrate and/or lipid and/or other antigen, including but not limited to one or more killed antigen(s) (e.g., a killed or completely inactive virus) or a live attenuated antigen (e.g., an attenuated virus). In some embodiments, the immunogenic composition(s) and/or vaccine(s) improve immune responses to any antigen regardless of the antigen source or its function.

As referred to herein, a “vector” carries a genetic code, or a portion thereof, for an antigen, however it is not the antigen itself. In an exemplary aspect, a vector can include a viral vector or bacterial vector. As referred to herein an “antigen” means a substance that induces a specific immune response in a subject, including humans and/or animals. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. In various aspects, the antigen is a virus, bacterium, a subunit of an organism, an auto-antigen, or a cancer antigen. In the context of a viral vector, an expressed heterologous antigen is an antigen derived from a different pathogen than the virus used for the viral vector.

As used herein, “ED₅₀” refers to 50% Effective Dilution, which is the reciprocal of the dilution of a serum sample that results in 50% neutralization of a toxin (e.g. anthrax lethal toxin) and it is determined as the reciprocal of the dilution corresponding to the inflection point (‘c’ parameter) of a 4-parameter logistic fir of the curve.

As used herein, “NF₅₀” refers to 50% Neutralization Factor, which is the quotient of the ED₅₀ of the test sample and the ED₅₀ of the reference serum. The NF50 serves as a relative measure of toxin neutralization and is less influenced by the day to day variability of an assay.

As used herein, TNA refers to toxin neutralization assay, wherein the TNA assay is designed to measure and quantify the functional ability of serum to neutralize B. anthracis LT activity using an in vitro cytotoxicity assay. The assay colorimetrically determines cell viability using a tetrazolium salt, 3-[4, 5-dimethylthiazol-2-yl]-2, 5-diphenyltetrazolium bromide (MTT) as the reporter, or signal system. The serum-mediated neutralization of anthrax LT manifests as a suppression of cytotoxicity and hence preservation of cell viability.

Immunogenic Compositions and Vaccines

Provided herein are pharmaceutically acceptable compositions (which may also be referred to as formulations) suitable and/or configured for intranasal administration to a mammalian subject and that are configured to induce an immune response against, or treat an infection thereof, an antigen (e.g., an immunogen), and optionally induce a protective immune response (i.e., as a vaccine). In some embodiments, the pharmaceutical formulation is an immunogenic composition that upon administration induces an immune response against an antigen in a mammalian subject). In some embodiments, the pharmaceutical formulation is an immunogenic composition that upon administration induces an anti-viral immune response that treats and/or protects against a respiratory pathogen and/or ameliorates symptoms of a respiratory infection in a mammalian subject. In some embodiments, the pharmaceutical formulation is a vaccine or therapeutic composition configured to induce a protective immune response in a mammalian subject, which is protective against foreign infectious agents, such as bacterial, viral or fungal pathogens, or in certain embodiments induce or stimulate a protective response against aberrant cell growth (e.g. cancer).

In certain embodiments, the present composition is an immunogenic composition (e.g., vaccine), that in some embodiments can comprise a vector (e.g., one or more vectors expressing a pathogenic antigen such as adenoviral (Ad) vectored immunogenic composition (e.g., vaccine)), a live attenuated pathogen (e.g. influenza virus that does not lead to a productive infection), killed or purified antigen (e.g., influenza antigen without active (i.e., infectious) virus; one or more immunogenic antigens induce an immune response in vivo without inducing infection), or live virus (e.g., FluMist® quadrivalent influenza immunogenic composition (e.g., vaccine); combination of four influenza viruses which do not replicate efficiently at normal body temperature (e.g. cold-adapted, live attenuated or temperature sensitive strains)). In certain embodiments, the immunogenic composition (e.g., vaccine) may comprise a viral vector that does not express a heterologous antigen, and in exemplary embodiments the viral vector may be replication deficient. See U.S. Pat. No. 9,605,275 (incorporated herein by reference in its entirety), wherein an empty adenoviral vector (AdE) rapidly induces a protective immune response against respiratory pathogens. In embodiments, the respiratory pathogens are selected from influenza and anthrax infections. In embodiments, use of AdE in the present methods induces a protective immune response against coronavirus, including SARS-Cov-2.

Moreover, the present compositions are configured for intranasal administration (e.g. liquid, spray, aerosol) but are not limited to inducing an immune response (e.g., a protective immune response) against a respiratory pathogen. In other words, intranasal administration provides a route of administration for inducing a systemic and/or protective response (e.g., as a vaccine) for any pathogen.

In certain embodiments the mammalian subject is a human subject. In some embodiments, the instant formulations comprise an effective amount of at least 10⁷ (at least 10⁸, at least 10⁹, at least 10¹⁰ or at least 10¹¹) viral particle (vp) of replication deficient adenovirus vector that contains and expresses a heterologous antigen from a pathogen, the DNA coding for the antigen having been codon optimized for the human subject and a pharmaceutically acceptable diluent or carrier. In exemplary embodiments the formulation is an anthrax immunogenic composition (e.g., vaccine) pharmaceutical formulation. In other exemplary embodiments the formulation is an influenza immunogenic composition (e.g., vaccine) pharmaceutical formulation. In certain embodiments the formulation is a coronavirus immunogenic composition (e.g., vaccine) pharmaceutical formulation. In certain embodiments the formulation is a respiratory pathogen immunogenic composition (e.g., vaccine) pharmaceutical formulation. In certain embodiments, the adenoviral vector is present in a formulation buffer comprising Tris HCl (pH 7.4), histidine, sucrose, sodium chloride, magnesium chloride, polysorbate 80, ethylenediaminetetraacetic acid, and ethanol.

In exemplary embodiments provided herein is an anthrax immunogenic composition (e.g., vaccine)) pharmaceutical formulation suitable for intranasal administration to a human subject, comprising: an effective amount of at least 10⁷ viral particles (vp) of replication deficient adenovirus vector that contains and expresses B. anthracis PA antigen codon optimized for the human subject; and, a pharmaceutically acceptable diluent or carrier.

In other exemplary embodiments provided herein is an influenza immunogenic composition (e.g., vaccine)) pharmaceutical formulation suitable for intranasal administration to a human subject, comprising: an effective amount of at least 10⁷ viral particles (vp), at least 10⁸ viral particles (vp), at least 10⁹ viral particles (vp), at least 10¹⁰ viral particles (vp), or in certain embodiments about 10¹¹ vp, which in some embodiments can be split between two nostrils providing a divided dose (e.g., 5×10¹⁰ vp per nostril), of replication deficient adenovirus vector that contains and expresses influenza virus hemagglutinin antigen (HA) codon optimized for the human subject; and, a pharmaceutically acceptable diluent or carrier.

In some embodiments, the present replication deficient adenovirus vector that contains and expresses influenza virus hemagglutinin antigen (HA) codon optimized for the human subject, may be combined with other influenza antigens (e.g. viral vector expressed antigens) to form a multivalent influenza pharmaceutical formulation. The other components may be included to induce a humoral response with antibodies to a different epitope than that presented in the instant adenoviral vector containing influenza virus hemagglutinin antigen. In other embodiments, the other component(s) may be included to induce a different arm of the immune system, such as cell-mediated or mucosal immune response to an influenza antigen.

In exemplary embodiments provided herein is a monovalent influenza pharmaceutical formulation suitable for a single dose intranasal administration to a human subject, comprising: an effective amount of at least 10⁸ viral particles (vp), at least 10⁹ viral particles (vp), at least 10¹⁰ viral particles (vp), or in some preferred embodiments about 10¹¹ vp, which in some embodiments can be split between two nostrils (e.g., 5×10¹⁰ vp per nostril), of replication deficient adenovirus vector that contains and expresses influenza virus hemagglutinin antigen codon optimized for the human subject; and, a pharmaceutically acceptable diluent or carrier. In certain embodiments, the effective amount induces a protective immune response configured to provide seroprotection to the human subject for at least 1 month (e.g., 28 days or 4 weeks), at least 2 months, at least 3 months, at least 6 months, at least 8 months, or at least 12 months against the influenza virus. In certain embodiments, the protective immune response comprises a combined mucosal, humoral and T cell response.

In other exemplary embodiments provided herein is an influenza pharmaceutical formulation suitable for a single dose intranasal administration to a human subject, comprising an effective amount of at least 10⁹ viral particles (vp), or in some preferred embodiments about 10¹¹ vp, which in some embodiments can be split between two nostrils (e.g., 5×10¹⁰ vp per nostril), of replication deficient adenovirus vector that contains and expresses influenza virus hemagglutinin antigen codon optimized for the human subject; and, a pharmaceutically acceptable diluent or carrier. In certain embodiments, the effective amount induces a protective immune response configured to provide seroprotection to the human subject of an HA1 antibody titer≥40 for at least 1 month (e.g. 28 days or 4 weeks), at least 2 months, at least 3 months, at least 6 months, at least 8 months, or at least 12 months against the influenza virus. In certain embodiments, the protective immune response comprises a combined mucosal, humoral and T cell response.

In certain embodiments, the non-replicating adenoviral viral vector is a human adenovirus. In alternative embodiments, the adenovirus is a bovine adenovirus, a canine adenovirus, a non-human primate adenovirus (e.g., chimpanzee), a chicken adenovirus, or a porcine or swine adenovirus. In exemplary embodiments, the non-replicating viral vector is a human adenovirus.

In some embodiments, the non-replicating adenoviral vectors are particularly useful for gene transfer into eukaryotic cells and immunogenic composition (e.g., vaccine) development, and in animal models.

In some embodiments, any adenoviral vector (Ad-vector) known to one of skill in art, and prepared for administration to a mammal, which may comprise and express an influenza antigen may be used in the compositions and with the methods of this application. Such Ad-vectors include any of those in U.S. Pat. Nos. 6,706,693; 6,716,823; 6,348,450; or US Patent Publ. Nos. 2003/0045492; 2004/0009936; 2005/0271689; 2007/0178115; 2012/0276138 (herein incorporated by reference in entirety).

In certain embodiments the recombinant adenovirus vector may be non-replicating or replication-deficient requiring complementing E1 activity for replication. In embodiments the recombinant adenovirus vector may include E1-defective, E3-defective, and/or E4-defective adenovirus vectors, or the “gutless” adenovirus vector in which viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. In exemplary embodiments, the recombinant adenovirus vector is an E1 and E3 defective vector.

The “gutless” adenovirus vector replication requires a helper virus and a special human 293 cell line expressing both E1a and Cre, a condition that does not exist in natural environment; the vector is deprived of viral genes, thus the vector as an immunogenic composition (e.g., vaccine) carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent. The adenovirus vector may also be used for invasive administration methods, such as intravenous, intramuscular, or subcutaneous injection.

With respect to dosages, routes of administration, formulations, adjuvants, and uses for recombinant viruses and expression products therefrom, compositions of the invention may be used for parenteral, topical, or mucosal administration, preferably by intradermal, subcutaneous, intranasal or intramuscular routes. When mucosal administration is used, it is possible to use oral, ocular or nasal routes. In exemplary embodiments, the present immunogenic compositions (e.g., vaccine) are administered intranasally. In exemplary and preferred embodiments, the present immunogenic compositions (e.g., vaccine) are administered intranasally wherein the subject during administration is in a modified sitting, supine, reclining, or other position wherein nostrils of the mammalian subject are tilted upward or elevated above a head of the mammalian subject.

The compositions (e. g., formulations) which comprise the adenovirus vector of interest, can be prepared in accordance with standard techniques well known to those skilled in the pharmaceutical or veterinary art. See Example 1 and 4a. Such formulations can be administered in dosages and by techniques well known to those skilled in the clinical arts taking into consideration such factors as the age, sex, weight, and the route of administration. The formulations can be administered alone (i.e., as the sole active agent(s)) or can be co-administered or sequentially administered with compositions, e.g., with “other” immunological composition, or attenuated, inactivated, recombinant immunogenic composition (e.g., vaccine) or therapeutic compositions thereby providing multivalent or “cocktail” or combination compositions of the invention and methods employing them. In some embodiments, the formulations may comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g., block or prevent metal-ion catalyzed free-radical oxidation).

The compositions (e.g., formulations) may be present in a liquid preparation for mucosal administration, e.g., oral, nasal, ocular, etc., formulations such as suspensions and, preparations for parenteral, subcutaneous, intradermal, intramuscular, intranasal, intravenous (e.g., injectable administration) such as sterile suspensions or emulsions. In such formulations the adenoviral vector may be in admixture with a suitable carrier, diluent, or excipient such as sterile water, physiological saline, viscosity enhancing excipients or the like. Certain specialized formulations for mucosal administration can be used, including mucoadhesives, mucosal penetrants and mucosal disruptants. The formulations can also be lyophilized or frozen. In some embodiments, the formulation is stored frozen and thawed to about room temperature (about 25° C.) prior to administration. The formulations can contain auxiliary substances such as wetting or emulsifying agents, pH buffering agents, adjuvants, preservatives, and the like, depending upon the route of administration and the preparation desired. The formulations can contain at least one adjuvant compound. In exemplary embodiments, the present immunogenic compositions (e.g., vaccines) are non-adjuvanted.

Standard texts, such as “REMINGTON'S PHARMACEUTICAL SCIENCE”, 17th edition, 1985, incorporated herein by reference, may be consulted to prepare suitable preparations, without undue experimentation.

In some embodiments, an effective amount (e.g., an amount that induces a protective immune response) of the adenoviral vector is at least 10⁷ viral particle (vp) of a replication deficient adenoviral vector containing and expressing B. anthracis PA antigen codon optimized for the human subject. As understood by one of skill in the art, codon optimization improves expression of heterologous genes in a host organism. The present B. anthracis PA antigen was codon optimized for a mammalian host, which includes a human subject.

In some embodiments, an effective amount (e.g., an amount that induces a protective immune response) of the adenoviral vector is at least 10⁸ viral particle (vp), or in some preferred embodiments about 10¹¹ vp, which in some embodiments can be split between two nostrils (e.g., 5×10¹¹ vp per nostril), of a replication deficient adenoviral vector containing and expressing influenza virus HA antigen codon optimized for the human subject. The present influenza virus HA antigen was codon optimized for a mammalian host, which includes a human subject. In some embodiments, the human subject has symptoms of SARS-CoV-2 infection, such as COVID-19, and preferably early COVID-19. Thus, in some embodiments, the antigen is an influenza antigen, a coronavirus antigen, or the composition comprises AdE, and the human being exhibits early coronavirus infectious disease (i.e., early COVID-19).

In certain embodiments, the present immunogenic composition (e.g., vaccine) comprises an effective amount of about 10⁷ viral particles (vp) of a replication deficient adenoviral vector. In exemplary embodiments, the present immunogenic composition (e.g., vaccine) comprises an effective amount of about 10⁸ viral particles (vp) of a replication deficient adenoviral vector. In certain other exemplary embodiments, the present immunogenic composition (e.g., vaccine) comprises an effective amount of about 10⁹ viral particles (vp) of a replication deficient adenoviral vector. In certain other exemplary embodiments, the present immunogenic composition (e.g., vaccine) comprises an effective amount of about 10¹⁰, or greater, viral particles (vp) of a replication deficient adenoviral vector. In certain other exemplary embodiments, the present immunogenic composition (e.g., vaccine) comprises an effective amount of about 10¹¹, or greater, viral particles (vp) of a replication deficient adenoviral vector. In some preferred embodiments using intranasal administration, the nominal dose can be split between two nostrils providing a divided dose (e.g., 5×10¹⁰ vp per nostril for a total dose of 10¹¹ vp).

In some embodiments, an effective amount (e.g., an amount that induces a protective immune response) of the adenoviral vector is at least 10⁹ viral particles (vp) (e.g., about 10¹⁰, 10¹¹ or more vp) of a replication deficient adenoviral vector containing and expressing influenza virus hemagglutinin antigen codon optimized for the human subject. In some embodiments, the present immunogenic compositions (e.g., monovalent influenza formulation) comprise an effective amount of about 10⁸ viral particles (vp) of a replication deficient adenoviral vector. In exemplary embodiments, the present immunogenic compositions (e.g., monovalent influenza formulation) comprises an effective amount of about 10⁹ viral particles (vp) of a replication deficient adenoviral vector. In certain other exemplary embodiments, the present immunogenic compositions (e.g., monovalent influenza formulation) comprises an effective amount of about 10¹⁰ viral particles (vp) of a replication deficient adenoviral vector. In some embodiments, the present immunogenic compositions (e.g., monovalent influenza formulation) comprises an effective amount of about 10¹¹ viral particles (vp) of a replication deficient adenoviral vector. In some preferred embodiments using intranasal administration, the nominal dose can be split between two nostrils (e.g., 5×10¹⁰ vp per nostril for a total nominal dose of 10¹¹ vp), administered to a human subject in the modified sitting, reclining or supine (dorsal) position (e.g., nostrils tilted back or above the head of the subject).

In some embodiments, the effective amount of adenoviral vector in the present immunogenic composition (e.g., vaccine) induces a protective immune response when the immunogenic composition (e.g., vaccine) is administered intranasally to a subject in a lying, supine, reclining, or other position wherein nostrils of the mammalian subject are elevated above a head of the mammalian subject. In some embodiments, the immune response provides protection against inhalation anthrax. In some embodiments, the immune response provides protection against influenza viral infection, including influenza viruses that cause seasonal or pandemic influenza infections. In some embodiments, the immune response provides protection against infection by SARS-CoV-2, or the development of or symptoms of COVID-19. In certain embodiments, the response to administration of an immunogenic composition of this disclosure that can provide immune protection against influenza viral infection (e.g., NasoVAX), can be administered to human subject with early coronavirus infection (preferably SARS-CoV-2 infection) and prevents clinical worsening in such a human subject. In embodiments, administration via the intranasal route of an immunogenic composition of this disclosure comprising a replication deficient adenoviral vector elicits/induces an innate immune response which provides protection against respiratory pathogens independent of the heterologous antigen (e.g. transgene) expressed by the viral vector, or even when AdE is administered.

In embodiments the immunogenic composition (e.g., vaccine) comprises an influenza antigen, a RSV antigen, a coronavirus antigen or fragment thereof (e.g. receptor binding domain (RBD) of spike (S) protein), a HIV antigen, a SIV antigen, a HPV antigen, a HCV antigen, a HBV antigen, a CMV antigen, a Staphylococcus antigen, or a fragment or epitope thereof. The influenza may be swine influenza, seasonal influenza, avian influenza, including, but not limited to influenza subtypes H1N1, H3N2 influenza, H7N9 influenza or H5N1 influenza. In certain embodiments, the immunogenic composition (e.g., vaccine) comprises an adenoviral vector encoding an influenza antigen, a coronavirus antigen, a RSV antigen, a HIV antigen, a SIV antigen, a HPV antigen, a HCV antigen, a HBV antigen, a CMV antigen, a Staphylococcus antigen, or a fragment or epitope thereof. The influenza may be swine influenza, seasonal influenza, avian influenza, H1N1 influenza or H5N1 influenza, including human-codon-optimized versions of these antigens. The coronavirus may be SARS-Cov, SARS-Cov-2, MERS, human coronavirus 229E (HCoV-229E), OC43 (HCoV-OC43), NL63, or HKU1. In embodiments, the coronavirus antigen may be spike protein or a fragment thereof (e.g. S1 domain or RBD of the S1 domain). In certain embodiments, the coronavirus antigen may be spike protein or a fragment thereof (e.g. S1 domain or RBD of the S1 domain) of the SARS-Cov-2 virus.

In certain embodiments the immunogenic composition (e.g., vaccine) comprises antigen from influenza hemagglutinin, influenza nuclear protein, influenza M2, tetanus toxin C-fragment, anthrax protective antigen, anthrax lethal factor, rabies glycoprotein, HBV surface antigen, HIV gp 120, HW gp 160, human carcinoembryonic antigen, malaria CSP, malaria SSP, malaria MSP, malaria pfg, mycobacterium tuberculosis HSP or a mutant thereof. In certain other embodiments, the immunogenic composition (e.g., vaccine) comprises an adenoviral vector encoding influenza hemagglutinin, influenza nuclear protein, influenza M1 or M2, influenza polymerase, tetanus toxin C-fragment, anthrax protective antigen, anthrax lethal factor, rabies glycoprotein, HBV surface antigen, HIV gp 120, HW gp 160, human carcinoembryonic antigen, Zika virus glycoprotein, or variants thereof, malaria CSP, malaria SSP, malaria MSP, malaria pfg, mycobacterium tuberculosis HSP or a mutant or variant thereof.

In exemplary embodiments, the present immunogenic composition (e.g., vaccine) comprises an adenoviral vector encoding B. anthracis PA antigen codon optimized for a mammalian host. In other exemplary embodiments, the present immunogenic composition (e.g., vaccine) comprises an adenoviral vector encoding influenza HA codon optimized for a mammalian host. In certain embodiments, the present immunogenic composition (e.g., vaccine) comprises an adenoviral vector encoding SARS-Cov-2 RBD domain of Spike protein codon optimized for a mammalian host. In certain embodiments, the present immunogenic composition (e.g., vaccine) comprises an adenoviral vector that does not encode a heterologous antigen of a respiratory pathogen.

In some embodiments, the effective amount of adenoviral vector in the present immunogenic composition (e.g., vaccine) induces a combined influenza-specific mucosal (as can be demonstrated via IgA measurement), humoral (as demonstrated via sera HA1; See FIGS. 10-13 (or microneutralization antibodies)) and cell mediated (as can be demonstrated via influenza HA antigen specific T cell activation) immune response in a human subject against influenza virus. In some embodiments, the serum antibodies are seroprotective for at least 1 month, at least 2 months, at least 3 months, at least 4 months, 6 months, at least 12 months, at least 13 month or at least 14 months. In some embodiments, the combined immune response provides protection against influenza A virus. In some embodiments, the combined immune response induced by the present monovalent influenza pharmaceutical formulation provides protection against Influenza B virus. In certain embodiments, the effective amount of adenoviral vector in the present immunogenic composition (e.g., vaccine) induces an innate immune response which can provide protection against respiratory pathogens.

In some embodiments, the influenza A virus and/or influenza B virus are “seasonal” influenza virus which cause seasonal epidemics, mostly during the winter months. In other embodiments, the influenza A virus is a “pandemic” influenza virus, which can cause widespread illness due to new and different antigenic epitopes present in the virus. influenza A viruses are divided into subtypes based on two proteins on the surface of the virus: the hemagglutinin (H) and the neuraminidase (N). There are at least 18 different hemagglutinin subtypes and at least 11 different neuraminidase subtypes. (H1 through H18 and N1 through N11 respectively.). influenza A viruses can be further broken down into different strains. Current subtypes of influenza A viruses found in people are influenza A (H1N1) and influenza A (H3N2) viruses. influenza B viruses are not divided into subtypes but can be further broken down into lineages and strains. Currently circulating influenza B viruses belong to one of two lineages: B/Yamagata and B/Victoria. Naming convention for influenza viruses includes multiple components and follows the approach: the antigenic type (e.g., A, B, C); the host of origin (e.g., swine, equine, chicken, etc. For human-origin viruses, no host of origin designation is given.); geographical origin (e.g., Denver, Taiwan, etc.); strain number (e.g., 15, 7, etc.); year of isolation (e.g., 57, 2009, etc.); and, for influenza A viruses, the hemagglutinin and neuraminidase antigen description in parentheses (e.g., (H1N1), (H5N1).

In exemplary embodiments, the present adenoviral vector comprises a genetic insert encoding the HA surface protein antigen from an A/California/04/2009(H1N1) virus. In certain embodiments, the present adenoviral vector contains and expresses a hemagglutinin antigen from an H1N1 influenza A virus subtype. In some embodiments, the present adenoviral vector contains and expresses a hemagglutinin antigen from an H3N2 influenza A virus subtype. In other embodiments, the present adenoviral vector contains and expresses a hemagglutinin antigen from an influenza B virus.

In some embodiments, the mammal is a companion or domesticated or food-producing or feed-producing or livestock or game or racing or sport animal such as a cow, a dog, a cat, a goat, a sheep, a rabbit, or a pig or a horse, or even fowl such as turkey, ducks or chicken. In exemplary embodiments the mammalian subject is a human.

Methods of Use

Provided herein is a method for transmucosal administration of a therapeutic dose of a present therapeutic/prophylactic/immunogenic composition (e.g., vaccine) configured to induce an immune response (e.g., a protective immune response as a vaccine) via intranasal administration wherein the subject is in a modified sitting, supine, reclining or other position wherein nostrils of the subject are pointing and/or tilting upwards and/or above head of the subject. In certain embodiments the present immunogenic compositions comprise an antigen from a pathogen to a mammalian subject.

In some embodiments, the method comprises inducing a protective immune response in a human subject via intranasal administration wherein the human subject, during administration, is in a modified sitting (head tilted back with nostrils elevated above head), reclining, supine, or other position wherein nostrils of the subject are pointing upwards or elevated above the head of the subject. In certain embodiments, the nostrils are elevated above a head or forehead of the subject. In some embodiments, the methods for transmucosal administration of a therapeutic dose of an immunogenic composition (e.g., vaccine) to a subject comprising an antigen from a pathogen to a mammalian subject, comprises administering intranasally to the subject an effective amount of the immunogenic composition (e.g., vaccine), wherein the subject during administration is in a modified sitting (e.g. head tilted back with nostrils elevated above head), supine, reclining, or other position wherein nostrils of the subject are elevated above a head or forehead of the subject.

In certain embodiments provided herein is a method for transmucosal administration of a therapeutic dose of a non-replicating viral vectored immunogenic composition (e.g., vaccine) to a human subject, wherein the method comprises administering intranasally to the human subject the immunogenic composition (e.g., vaccine) comprising an effective amount of at least 10⁷ viral particle (vp) of replication deficient adenovirus vector that contains and expresses a heterologous antigen codon optimized for the mammalian subject, wherein the subject during administration is in a modified sitting (e.g., head tilted back with nostrils positioned above the remainder of the face of the subject), supine, reclining or other position wherein nostrils of the subject are elevated above a head of the subject; whereby the therapeutic dose administered transmucosally induces a protective immune response. In embodiments, the method comprises administering an effective amount of at least 10⁸ viral particle (vp), at least 10⁹ viral particle (vp), at least 10¹⁰ viral particle (vp), of replication deficient adenovirus vector that contains and expresses a heterologous antigen codon optimized for the mammalian subject.

In certain embodiments provided herein is a method for transmucosal administration of a therapeutic dose of a non-replicating viral vectored immunogenic composition (e.g., vaccine) to a human subject, wherein the method comprises administering intranasally to the human subject the immunogenic composition (e.g., vaccine) comprising an effective amount of at least 10¹¹ viral particle (vp) of replication deficient adenovirus vector that contains and expresses a heterologous antigen codon optimized for the mammalian subject, wherein the subject during administration is in a modified sitting (head tilted back with nostrils elevated above head), supine, reclining or other position wherein nostrils of the subject are elevated above a head of the subject; whereby the therapeutic dose administered transmucosally induces a protective immune response.

In certain embodiments provided herein is a method for transmucosal administration of a therapeutic dose of a non-replicating viral vectored immunogenic composition (e.g., vaccine) to a human subject, wherein the method comprises administering intranasally to the human subject the immunogenic composition (e.g., vaccine) comprising an effective amount of at least 10⁷ viral particle (vp) of replication deficient adenovirus vector without containing and expressing (encoding) a heterologous antigen, wherein the subject during administration is in a modified sitting (head tilted back with nostrils elevated above head), supine, reclining or other position wherein nostrils of the subject are elevated above a head of the subject; whereby the therapeutic dose administered transmucosally induces a protective immune response. In embodiments, the protective immune response is an innate immune response and provides protection against respiratory pathogens. In embodiments, the method comprises administering an effective amount of at least 10⁸ viral particle (vp), at least 10⁹ viral particle (vp), or at least 10¹⁰ viral particle (vp), of replication deficient adenovirus vector that contains and expresses a heterologous antigen codon optimized for the mammalian subject.

In certain embodiments provided herein is a method for transmucosal administration of a therapeutic dose of a non-replicating viral vectored immunogenic composition (e.g., vaccine) to a human subject, wherein the method comprises administering intranasally to the human subject the immunogenic composition (e.g., vaccine) comprising an effective amount of at least 10¹¹ viral particle (vp) of replication deficient adenovirus vector without containing and expressing (encoding) a heterologous antigen, wherein the subject during administration is in a modified sitting (head tilted back with nostrils elevated above head), supine, reclining or other position wherein nostrils of the subject are elevated above a head of the subject; whereby the therapeutic dose administered transmucosally induces a protective immune response. In embodiments, the protective immune response is an innate immune response and provides protection against respiratory pathogens.

In certain embodiments, following administration wherein the subject was in a modified sitting (head tilted back with nostrils elevated above head), supine, reclining or other position wherein nostrils of the subject are elevated above a head of the subject, there is a post-dose hold period wherein the subject remains in the dosing positions (i.e., nostrils of the subject are elevated above the head of the subject). In embodiments, the post-dose hold period is from about 1 minute to about 35 minutes. In embodiments, the post-dose hold period is about 1 minute, about 2 minutes, about 3 minutes, about 4 minutes, about 5 minutes, about 7 minutes, about 10 minutes, about 12 minutes, about 15 minutes, about 17 minutes, about 20 minutes, about 22 minutes, about 25 minutes, about 27 minutes, about 30 minutes, or about 33 minutes.

In some embodiments, the adenoviral vector encodes B. anthracis PA antigen codon optimized for the human host. In certain embodiments, the adenoviral vector encodes influenza virus HA antigen codon optimized for the human host. In certain embodiments, the adenoviral vector encodes SARS-Cov-2 Spike protein, or fragment thereof (e.g. RBD domain of S protein). In certain other embodiments, the adenoviral vector does not encode a heterologous antigen of a respiratory pathogen, In some embodiments, administering the immunogenic composition (e.g., vaccine) to the human host intranasally when the subject is in a modified sitting (head tilted back with nostrils elevated above head), supine, reclining or other position wherein nostrils of the subject are elevated above the head of the subject induces a protective immune response (i.e., it acts as a vaccine), whereas intranasal administration of the immunogenic composition (e.g., vaccine) when the subject is in a normal sitting position with nostrils pointed down does not induce a protective immune response (i.e., it does not act as a vaccine).

In some embodiments, this disclosure provides methods of inducing a protective immune response in a human subject against influenza virus whereby the human subject is seroprotected within 15 days, or within 28 days post administration with an intranasal effective dose of about 10⁸ viral particles (vp) of the present replication deficient adenoviral vector encoding an influenza antigen. In certain embodiments, that protective immune response endures for at least 6 months, at least 12 months, at least 13 months or longer. In some embodiments, the methods comprise administering intranasally a single dose of an effective amount of at least 10⁸ viral particles (vp) of a replication deficient adenoviral vector containing and expressing influenza virus hemagglutinin antigen codon optimized for the human subject, wherein the administration induces sera antibodies. In some embodiments, the present monovalent influenza pharmaceutical formulation is used to provide protection against seasonal influenza virus. In certain other embodiments, the present monovalent influenza pharmaceutical formulation is used to provide protection against pandemic influenza virus. In some embodiments, the present methods provide protection against infection by influenza A virus subtypes. In certain embodiments, the present methods provide protection against infection by influenza A virus subtypes H1N1 and/or H3N2. In other embodiments, the present methods provide protection against infection by Influenza B virus. In some embodiments, the seroprotection lasts at least about 13 months. In certain embodiments, the seroprotection lasts at least about 14 months, or longer.

In some embodiments, the step of administering a single dose of a present (monovalent) influenza pharmaceutical formulation induces HAI antibodies at a titer of 40 or greater within 28 days post administration and endures for at least 2 months, at least 3 months, at least 4 months, at least 6 months, at least 12 months, at least 13 months, at least 14 months or longer. In embodiments the titer of HAI antibodies in a human subject 28 days post vaccination (administration of the present influenza pharmaceutical formulation) is at least 50, at least 60, at least 70, at least 80, at least 90, or at least 100.

In certain embodiments, this disclosure provides methods of inducing a protective immune response in a human subject against coronavirus. In certain embodiments the present immunogenic compositions comprise a replication defective adenoviral vector comprising an expression cassette comprising a coding sequence encoding at least one coronavirus antigen or at least one immunogenic fragment thereof. In certain other embodiments, the present immunogenic compositions comprise a replication defective adenoviral vector without encoding at least one coronavirus antigen. In certain embodiments the mammalian subject is a human being and the coronavirus antigen is from SARS-CoV-2. In some embodiments, the mammalian subject is a human being infected by SARS-CoV-2 (e.g., a hospitalized human being). In some embodiments, the SARS-CoV-2 immunogenic composition can be used to treat SARS-CoV-2 infection (e.g., in such an infected and/or hospitalized human being).

In embodiments, the present immunogenic composition is used to provide treatment and/or protection against seasonal coronavirus. In certain other embodiments, the present immunogenic composition is used to provide treatment and/or protection against pandemic SARS-CoV-2. In embodiments, the treatment begins within about 24 hours via an innate immune response. In certain other embodiments, the present immunogenic composition is used to provide protection against SARS-CoV-2. In embodiments, the seroprotection lasts at least about 1 month, 2 months, 4 months, 6 months, 8 months, 10 months, 12 month or at least about 13 months.

In certain embodiments, this disclosure provides methods of inducing a protective immune response in a human subject against a respiratory pathogen (e.g. influenza, B. anthracis, coronavirus) via administration of a single dose. In alternative embodiments, provided herein is a method for inducing an immune response against respiratory pathogens wherein the method comprises administering at least a prime and boost dose of a present immunogenic composition/formulation/dosage. In certain embodiments, the boost dose is administered about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks or 52 weeks after administration of the prime dose.

In embodiments, the prime and boost dose are administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

In embodiments, the prime boost doses are homologous, meaning they compromise the same immunogenic composition. In certain embodiments, the methods and compositions provided include administering a heterologous vaccine prime dose and boost dose leading to an induction of an immune response where “heterologous” means a prime dose that is different than a boost dose. For example, an immunogenic composition (e.g. adenoviral vector encoding a heterologous antigen) may be used to induce a rapid (innate) immune response against one or more respiratory pathogens (e.g. influenza) followed by a boost dose of an immunogenic compositions (e.g. adenoviral vector with or without encoding a heterologous antigen), wherein the encoded heterologous antigen would be different between the prime dose and the boost dose. The first composition can be administered first to prime the immune response locally or systemically and the second vaccine such as the adenovirus can be administered mucosally as a booster to “pull” the primed immune cells locally and restimulated them in an antigen specific manner.

In embodiments, the first or second immunogenic composition is administered as a prime and boost dose administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

Dosage of the immunogenic composition (e.g. vaccine) when used with or without an adjuvant may range from about 10⁷ to about 10¹² infectious unit or plaque forming unit (ifu or pfu), or the dosage unit may be a viral particle (vp), wherein 1 vp equals about 1-100 ifu or pfu. In one embodiment the dose of immunogenic composition administered to the mammalian subject is about, or at least about, 10⁷ vp. In another aspect the dose of immunogenic composition administered to the mammalian subject is about, or at least about, 10⁸ vp. In yet another aspect, the dose of immunogenic composition administered to the mammalian subject is about, or at least about, 10⁹ vp. In another aspect the dose of immunogenic composition administered to the mammalian subject is about, or at least about, 10¹⁰ vp. In another aspect the dose of immunogenic composition administered to the mammalian subject is about, or at least about, 10¹¹ vp. In another aspect the dose of immunogenic composition administered to the mammalian subject is about, or at least about, 10¹² vp.

In embodiments, for (trans)mucosal administration compositions may be in a form and dispensed by a squeeze spray dispenser, pump dispenser, multi-dose dispenser, dropper-type dispenser, atomizer or aerosol dispenser. Aerosols are usually under pressure by means of a hydrocarbon. Pump dispensers may preferably dispense a metered dose or, a dose having a particular particle size.

Thus, in some embodiments, this disclosure provides methods for transmucosal administration of a therapeutic dose of an immunogenic composition (e.g., vaccine) comprising an antigen from a pathogen to a mammalian subject, wherein the method comprises: administering intranasally to the mammalian subject an effective amount of the immunogenic composition (e.g., vaccine), wherein the subject during administration is in a sitting, reclining, supine, or other position wherein nostrils of the mammalian subject are tilted upward; whereby the therapeutic dose administered transmucosally induces a protective immune response. In some embodiments, the immunogenic composition (e.g., vaccine) comprises a non-replicating viral vector that encodes a heterologous antigen. In some embodiments, the non-replicating viral vector is an adenovirus vector. In some embodiments, the adenovirus is a human adenovirus. In some embodiments, the adenovirus vector is an E1 and E3 deleted adenovirus vector. In some embodiments, the adenovirus is a bovine adenovirus, a canine adenovirus, a non-human primate adenovirus, a chicken adenovirus, or a porcine or swine adenovirus. In some embodiments, the effective amount is at least 10⁷ viral particle (vp) of E1 and/or E3 deleted adenovirus. In some embodiments, the effective amount is at least 10⁸ viral particle (vp) of E1 and/or E3 deleted or disrupted adenovirus. In some embodiments, the effective amount is at least 10⁹ viral particle (vp) of E1 and/or E3 deleted or disrupted adenovirus. In some embodiments, the mammalian subject is administered intranasally the immunogenic composition (e.g., vaccine) comprising an effective amount of at least 10⁷ viral particle (vp) of replication deficient adenovirus vector that contains and expresses a heterologous antigen codon optimized for the mammalian subject.

In some embodiments, the pathogen is a respiratory pathogen. In some embodiments, the pathogen is a virus. In some embodiments, the virus is an orthomyxovirus, a paramyxovirus, a rhinovirus or a coronavirus. In some embodiments, the virus is an influenza virus, a respiratory syncytial virus (RSV), a common cold virus or a measles virus. In some embodiments, the common cold virus is a rhinovirus or a coronavirus. In some embodiments, the pathogen is a bacterium. In some embodiments, the bacterium is selected from the group consisting of Bacillus, Mycobacterium, Staphylococcus, Streptococcus, Pseudomonas, Klebsiella, Haemophilus, and Mycoplasma. In some embodiments, the bacterium is Bacillus anthracis. In some embodiments, the antigen is Bacillus anthracis protective antigen. In some embodiments, the B. anthracis protective antigen is PA83. In some embodiments, the antigen is Bacillus anthracis lethal factor. In some embodiments, the pathogen is a fungus. In some embodiments, the fungus is Aspergillus. In some embodiments, the antigen is an influenza antigen, optionally wherein: the influenza antigen is from a seasonal influenza virus, an Influenza A virus, and/or an Influenza B virus; and/or the influenza antigen is a hemagglutinin (HA) surface protein antigen.

In some embodiments, the therapeutic dose of the immunogenic composition (e.g., vaccine) comprises a monovalent influenza pharmaceutical formulation suitable for a single dose intranasal administration to a human subject, the immunogenic composition (e.g., vaccine) comprising an effective amount of at least 10⁸ viral particles (vp) of replication deficient adenovirus vector that contains and expresses influenza virus hemagglutinin antigen codon optimized for the human subject, wherein the effective amount induces a protective immune response configured to provide seroprotection to the human subject of an HA1 antibody titer≥40 within 28 days post administration against the influenza virus, and a pharmaceutically acceptable diluent or carrier. In some embodiments, the formulation is configured to provide seroprotection to the human subject of an HA1 antibody titer≥40 for at least 2 months, at least 3 months, at least 4 months, at least 6 months, at least 8 months, at least 10 months, at least 12 months against the influenza virus, optionally wherein the HA1 antibody titer is at least 50 and/or within 28 days post administration. In some embodiments, the effective amount is at least 10⁹ or at least 10¹⁰ viral particles (vp).

In some embodiments, the formulation does not comprise an adjuvant. In some embodiments, the influenza virus HA antigen is from an Influenza A virus, optionally wherein the Influenza A virus is subtype H1N1 and/or subtype H3N2. In some embodiments, the method induces a seroprotective immune response lasting for at least 2 months, at least 3 months, at least 4 months, at least 6 months, at least 8 months, at least 10 months, at least 12 months, at least 13 months or at least 14 months. In some embodiments, the method induces a combined mucosal, humoral and T cell protective immune response provides protection against Influenza A virus and Influenza B virus subtype. In some embodiments, the mammalian subject is a companion animal, a domesticated animal, a food-or feed-producing animal, a livestock animal, a game animal, a racing animal, or a sport animal. In some embodiments, the mammalian subject is a cow, a horse, a rabbit, a dog, a cat, a goat, a sheep, or a pig. In some embodiments, the mammalian subject is a human, optionally wherein the human is an adult. In some embodiments, the human subject can be one infected by SARS-CoV-2 and/or exhibits symptoms of COVID-19. In preferred embodiments, the method can prevent clinical worsening in a human subject with early coronavirus infection (preferably SARS-CoV-2 infection). Other embodiments of the same are also contemplated herein, as would be understood by those of ordinary skill in the art.

In some embodiments, this disclosure provides methods for transmucosal administration of a therapeutic dose of a non-replicating viral vectored immunogenic composition (e.g., vaccine) to a mammalian subject, wherein the method comprises administering intranasally to the mammalian subject the immunogenic composition (e.g., vaccine) comprising an effective amount of at least 10⁷ viral particle (vp), at least about 10⁸ vp, at least about 10⁹ vp, at least about 10¹⁰ vp, or at least about 10¹¹ vp, of replication deficient adenovirus vector that contains and expresses a heterologous antigen codon optimized coding sequence for the mammalian subject, wherein the subject during administration is in a sitting, reclining, supine, or other position wherein nostrils of the mammalian subject are tilted upward; and, whereby the therapeutic dose administered transmucosally induces a protective immune response (i.e., it acts as a vaccine). In preferred embodiments, the “heterologous antigen” is heterologous with respect to the infectious agent for which the immunogenic composition is intended to provide protection. For instance, the use of NasoVAX disclosed herein to provide protection against infection by SARS-CoV-2, or the symptoms of COVID-19, would be considered the use of a heterologous antigen (i.e., an influenza antigen being used to prevent, treat, and/or alleviate the symptoms of SARS-CoV-2 infection, including COVID-19). In some embodiments, the coding sequence encodes at least one antigen of a pathogen selected from the group consisting of a respiratory pathogen; virus; an orthomyxovirus; a paramyxovirus; a rhinovirus; a coronavirus; an influenza virus that is optionally a seasonal influenza virus, an Influenza A virus, and/or an Influenza B virus; a respiratory syncytial virus (RSV); a common cold virus; a measles virus; a bacterium optionally selected from the group consisting of Bacillus such as Bacillus anthracis, Mycobacterium, Staphylococcus, Streptococcus, Pseudomonas, Klebsiella, Haemophilus, and Mycoplasma; a fungus that is optionally Aspergillus. In some embodiments, the antigen is Bacillus anthracis protective antigen, optionally PA83 and/or Bacillus anthracis lethal factor. In some embodiments, the antigen is an influenza antigen, optionally a hemagglutinin (HA) surface protein antigen, and: the composition is configured to provide seroprotection to the human subject of an HA1 antibody titer≥40 within 28 days post administration against the influenza virus, optionally wherein the HA1 antibody titer is at least 50 and/or within 28 days post administration; the effective amount is at least 10⁹ or at least 10¹⁰ viral particles (vp); the composition does not comprise an adjuvant; the influenza virus HA antigen is from an Influenza A virus, optionally wherein the Influenza A virus is subtype H1N1 and/or subtype H3N2; the method induces a seroprotective immune response lasting for at least 2, 3, 4, 6, 8, 10, 12, 13 or 14 months; and/or, the method induces a combined mucosal, humoral and T cell protective immune response provides protection against Influenza A virus and Influenza B virus subtype.

In some embodiments, the methods disclosed herein include administration of the immunogenic composition in two doses split approximately evenly between two nostrils of the mammalian subject while the mammalian subject is in the dorsal (i.e., supine) position. In some embodiments, the mammalian subject is in the supine position during administration and remains in the supine position for at least about 30 minutes following administration. In some embodiments, the method delivers an intranasal nominal dose of at least about 1×10¹¹ virus particles (vp) (e.g., NasoVAX drug product). In some embodiments, the method includes administering about a 0.5 mL dose of the NasoVAX drug product (preferably thawed to room temperature, about 25° C.) is administered to deliver 0.25 mL of an intranasal spray to each nostril of the mammalian subject. In some embodiments, the antigen (i.e., of a pathogen or heterologous antigen (heterologous as to the infectious agent for which the immunogenic composition is intended to produce an immune response against)) is an influenza antigen, optionally wherein the influenza antigen is the hemagglutinin (HA) surface antigen. In some embodiments, the antigen is an influenza antigen and mammalian subject is a human being with early coronavirus infectious disease (i.e., early COVID-19). In some embodiments, such methods prevent clinical worsening in the human being, wherein the clinical worsening is at least about a 4.0% decrease from baseline in resting SpO₂ as determined by mobile pulse oximetry on two consecutive measurements following administration. In some embodiments, the clinical worsening can be determined by measuring at least one characteristic selected from the group consisting of maximal severity of COVID-19 after treatment as assessed by average decrease in resting SpO₂ from baseline resting SpO₂ at screening during the 14 days following administration, average increase in resting pulse rate from baseline resting pulse rate at screening during the 14 days following administration, proportion of patients requiring hospitalization on Day 42 following administration according to the level of oxygen supplementation required, and/or all-cause mortality through Day 42 following administration. In preferred embodiments, the mammalian subject to whom the immunogenic composition is administered does not exhibit a serious adverse event following administration.

In some embodiments, this disclosure provides method(s) for transmucosal administration of a therapeutic dose of an immunogenic composition comprising an antigen from a pathogen to a mammalian subject, wherein the method comprises: administering intranasally to the mammalian subject an effective amount of the immunogenic composition, wherein the subject during administration is in a modified sitting, reclining, supine, or other dosing position wherein nostrils of the mammalian subject are tilted upward; whereby the therapeutic dose administered transmucosally induces a protective immune response; optionally wherein the immunogenic composition is a vaccine. In some embodiments, this disclosure provides method(s) for inducing in a mammalian subject a protective immune response, comprising: intranasally administrating an effective amount of a therapeutic dose of an immunogenic composition to the mammalian subject, wherein the immunogenic composition comprises an antigen from a pathogen to the mammalian subject, wherein during the intranasal administration the mammalian subject is in a modified sitting, reclining, or supine position, wherein nostrils of the mammalian subject are tilted upward; and, instructing or otherwise keeping the mammalian subject to remain in the modified sitting, reclining, or supine position for about 1 to about 35 minutes after the intranasal administration, whereby the therapeutic dose administered intranasally induces a protective immune response in the mammalian subject. In some embodiments, this disclosure provides method(s) for vaccinating a patient (a type of mammalian subject) against viral infection, comprising: intranasally administrating an effective amount of a therapeutic dose of an immunogenic composition to the patient, wherein the immunogenic composition comprises an antigen from a pathogen to the patient, wherein during the intranasal administration the patient is in a modified sitting, reclining, or supine position, wherein nostrils of the mammalian subject are tilted upward; and, instructing or otherwise keeping the patient to remain in the modified sitting, reclining, or supine position for about 1 to about 35 minutes after the intranasal administration, whereby the therapeutic dose administered intranasally induces a protective immune response in the patient. In some embodiments, this disclosure provides method(s) for treating viral infection in a patient (a type of mammalian subject), comprising: intranasally administrating an effective amount of a therapeutic dose of an immunogenic composition to the patient, wherein the immunogenic composition comprises an antigen from a pathogen to the patient, wherein during the intranasal administration the patient is in a modified sitting, reclining, or supine position, wherein nostrils of the patient are tilted upward; and, instructing or otherwise keeping the patient to remain in the modified sitting, reclining, or supine position for about 1 to about 35 minutes after the intranasal administration, whereby the therapeutic dose administered intranasally induces a protective immune response in the patient. In some embodiments, the nostrils of the mammalian subject are elevated above the head of the mammalian subject. In some embodiments, the methods can further comprise instructing or otherwise keeping the mammalian subject to remain in the dosing position during a post-dose hold period selected from about 5 minutes to about 30 minutes. In some embodiments, less than about 20% of the therapeutic dose leaves the nasal cavity via drainage externally. In some embodiments, the immunogenic composition can be administered as a single dose. In some embodiments, the immunogenic composition can be administered as a divided dose in each nostril, optionally wherein the divided dose is administered using a multidose intranasal delivery device. In some embodiments, the immunogenic composition can be provided as a prime dose and a boost dose, optionally wherein the immunogenic composition is provided as a dosing regimen in combination with a heterologous dose as a prime dose or boost dose. In some embodiments, the immunogenic composition comprises a non-replicating viral vector that encodes and expresses a heterologous antigen. In some embodiments, the immunogenic composition comprises a non-replicating viral vector without encoding a heterologous antigen. In some embodiments, the immunogenic composition comprises a non-replicating adenovirus vector. In some embodiments, the adenovirus can be a human adenovirus. In some embodiments, the adenovirus can be selected from the group consisting of a bovine adenovirus, a canine adenovirus, a non-human primate adenovirus, a chicken adenovirus, or a porcine and a swine adenovirus. In some embodiments, the adenovirus vector can be an E1 and E3 deleted adenovirus vector. In some embodiments, the effective amount can be selected from the group consisting of at least 10⁷ viral particle (vp) of E1 and/or E3 deleted adenovirus, at least 10⁸ viral particle (vp) of E1 and/or E3 deleted or disrupted adenovirus, at least 10⁹ viral particle (vp) of E1 and/or E3 deleted or disrupted adenovirus, at least 10¹⁰ viral particle (vp) of E1 and/or E3 deleted or disrupted adenovirus, and at least 10¹¹ viral particle (vp) of E1 and/or E3 deleted or disrupted adenovirus. In some embodiments, the mammalian subject can be administered intranasally the immunogenic composition comprising an effective amount of at least 10⁷ viral particle (vp) of replication deficient adenovirus vector that contains and expresses a heterologous antigen codon optimized for the mammalian subject. In some embodiments, the pathogen can be a respiratory pathogen. In some embodiments, the pathogen can be a virus that, in some embodiments, can be selected from the group consisting of an orthomyxovirus, a paramyxovirus, a rhinovirus and a coronavirus. In some embodiments, the coronavirus can be selected from the group consisting of SARS-Cov, SARS-Cov-2, MERS, human coronaviruses 229E (HCoV-229E), OC43 (HCoV-OC43), NL63, and HKU1. In some embodiments, the virus is selected from the group consisting of an influenza virus, a respiratory syncytial virus (RSV), a common cold virus and a measles virus. In some embodiments, the common cold virus can be a rhinovirus or a coronavirus. In some embodiments, the pathogen can be a bacterium. In some embodiments, the bacterium can be selected from the group consisting of Bacillus, Mycobacterium, Staphylococcus, Streptococcus, Pseudomonas, Klebsiella, Haemophilus, and Mycoplasma. In some embodiments, the bacterium can be Bacillus anthracis. In some embodiments, the antigen is Bacillus anthracis protective antigen. In some embodiments, the Bacillus anthracis protective antigen can be PA83. In some embodiments, the antigen can be Bacillus anthracis lethal factor. In some embodiments, the pathogen can be a fungus. In some embodiments, the fungus can be Aspergillus. In some embodiments, the antigen can be an influenza antigen selected from the group consisting of a seasonal influenza virus antigen, an influenza A virus antigen, an influenza B virus antigen. In some embodiments, the antigen can be a hemagglutinin (HA) surface protein influenza antigen, or fragment thereof.

In some embodiments, the therapeutic dose of the immunogenic composition administered as disclosed herein comprises a monovalent influenza pharmaceutical formulation suitable for a single dose intranasal administration to a human subject, the immunogenic composition comprising: an effective amount of at least 10⁸ viral particles (vp) of replication deficient adenovirus vector that contains and expresses influenza virus hemagglutinin antigen codon optimized for the human subject, wherein the effective amount induces a protective immune response configured to provide seroprotection to the human subject of a hemagglutination inhibition assay (HAI) antibody titer≥40 within 28 days after administration against the influenza virus; and, a pharmaceutically acceptable diluent or carrier. In some embodiments, this disclosure provides: a) the formulation is configured to provide seroprotection to the human subject of an HAI antibody titer≥40 within 28 days post administration against the influenza virus, optionally wherein the HAI antibody titer is at least 50 and/or within 28 days post administration; b) the effective amount is at least 10⁹ or at least 10¹⁰ viral particles (vp); c) the formulation does not comprise an adjuvant; d) the influenza virus HA antigen is from an influenza A virus, optionally wherein the influenza A virus is subtype H1N1 and/or subtype H3N2; e) the method induces a seroprotective immune response lasting for at least 12 months; f) the method induces a combined mucosal, humoral and T cell protective immune response and provides protection against influenza A virus and influenza B virus subtype; and/or, g) the immune response is measured by the haemagglutination inhibition assay (HAI); as well as methods for using the same.

In some embodiments, this disclosure provides method(s) for transmucosal administration of a therapeutic dose of a non-replicating viral vectored immunogenic composition to a mammalian subject, wherein the method comprises: administering intranasally to the mammalian subject an immunogenic composition comprising an effective amount of at least 10⁸ viral particle (vp) of replication deficient adenovirus vector, with or without containing and expressing a heterologous antigen codon optimized for the mammalian subject, wherein the subject during administration is in a modified sitting, reclining, supine, or other dosing position wherein nostrils of the mammalian subject are tilted upward; whereby the therapeutic dose administered transmucosally induces a protective immune response; optionally wherein the immunogenic composition is a vaccine.

In some embodiments, this disclosure provides method(s) for inducing in a mammalian subject a protective immune response, comprising: intranasally administrating an effective amount of a therapeutic dose of an immunogenic composition to the mammalian subject, wherein the immunogenic composition comprises an effective amount of at least 10⁸ viral particle (vp) of replication deficient adenovirus vector, with or without encoding a heterologous antigen from a pathogen to the mammalian subject, wherein during the intranasal administration the mammalian subject is in a modified sitting, reclining, or supine position, wherein nostrils of the mammalian subject are tilted upward; and, instructing or otherwise keeping the mammalian subject to remain in the modified sitting, reclining, or supine position for about 1 minute to about 30 minutes after the intranasal administration, whereby the therapeutic dose administered intranasally induces a protective immune response in the mammalian subject. In some embodiments, this disclosure provides method(s) for vaccinating a patient against viral infection, comprising: intranasally administrating an effective amount of a therapeutic dose of an immunogenic composition to the patient, wherein the immunogenic composition comprises an effective amount of at least 10⁸ viral particle (vp) of replication deficient adenovirus vector, with or without encoding a heterologous antigen from a pathogen to the mammalian subject, wherein during the intranasal administration the patient is in a modified sitting, reclining, or supine position, wherein nostrils of the patient are tilted upward; and, instructing or otherwise keeping the patient to remain in the modified sitting, reclining, or supine position for about 1 minute to about 30 minutes after the intranasal administration, whereby the therapeutic dose administered intranasally induces a protective immune response in the patient (a type of mammalian subject). In some embodiments, this disclosure provides method(s) for treating viral infection in a patient, comprising: intranasally administrating an effective amount of a therapeutic dose of an immunogenic composition to the patient, wherein the immunogenic composition comprises an effective amount of at least 10⁸ viral particle (vp) of replication deficient adenovirus vector, with or without encoding a heterologous antigen from a pathogen to the mammalian subject, wherein during the intranasal administration the patient is in a modified sitting, reclining, or supine position, wherein nostrils of the patient are tilted upward; and, instructing or otherwise keeping the patient to remain in the modified sitting, reclining, or supine position for about 1 minute to about 30 minutes after the intranasal administration, whereby the therapeutic dose administered intranasally induces a protective immune response in the patient (a type of mammalian subject). In some embodiments, the nostrils of the mammalian subject can be elevated above the head of the mammalian subject. In some embodiments, the methods can further comprise instructing or otherwise keeping the mammalian subject to remain in the dosing position during a post-dose hold period selected from about 5 minutes to about 30 minutes. In some embodiments, less than about 20% of the pharmaceutical composition leaves the nasal cavity via drainage externally. In some embodiments, the immunogenic composition can be administered as a single dose. In some embodiments, the immunogenic composition can be administered as a divided dose in each nostril, which can optionally be administered using a multidose intranasal delivery device. In some embodiments, the immunogenic composition can be provided as a prime dose and a boost dose. In some embodiments, the immunogenic composition can be provided as a dosing regimen in combination with a heterologous dose as a prime dose or boost dose. In some embodiments, the protective immune response can be induced within about 24 hours. In some embodiments, the protective immune response can be induced within about 1 or 2 days. In some embodiments, the adenovirus can be a human adenovirus. In some embodiments, the adenovirus can be selected from the group consisting of a bovine adenovirus, a canine adenovirus, a non-human primate adenovirus, a chicken adenovirus, or a porcine and a swine adenovirus. In some embodiments, the adenovirus vector can be an E1 and E3 deleted adenovirus vector. In some embodiments, the effective amount can be selected from the group consisting of at least 10⁹ viral particle (vp) of E1 and/or E3 deleted or disrupted adenovirus, at least 10¹⁰ viral particle (vp) of E1 and/or E3 deleted or disrupted adenovirus, and at least 10¹¹ viral particle (vp) of E1 and/or E3 deleted or disrupted adenovirus. In some embodiments, the heterologous antigen can be from a respiratory pathogen. In some embodiments, the respiratory pathogen can be a virus. In some embodiments, the virus can be selected from the group consisting of an orthomyxovirus, a paramyxovirus, a rhinovirus and a coronavirus. In some embodiments, the coronavirus can be selected from the group consisting of SARS-Cov, SARS-Cov-2, MERS, human coronavirus 229E (HCoV-229E), OC43 (HCoV-OC43), NL63, and HKU1. In some embodiments, the virus can be selected from the group consisting of an influenza virus, a respiratory syncytial virus (RSV), a common cold virus and a measles virus. In some embodiments, the common cold virus can be a rhinovirus or a coronavirus. In some embodiments, the respiratory pathogen can be a bacterium selected from the group consisting of Bacillus, Mycobacterium, Staphylococcus, Streptococcus, Pseudomonas, Klebsiella, Haemophilus, and Mycoplasma. In some embodiments, the respiratory pathogen can be a fungus that is optionally Aspergillus. In some embodiments, the heterologous antigen can be Bacillus anthracis protective antigen, optionally PA83 and/or Bacillus anthracis lethal factor. In some embodiments, the heterologous antigen can be an influenza antigen, optionally a hemaglutinnin (HA) surface protein antigen, and: a) the composition is configured to provide seroprotection to a mammalian subject of an HA1 antibody titer≥40 within 28 days post administration against the influenza virus, optionally wherein the HA1 antibody titer is at least 50 and/or within 28 days post administration; b) the effective amount is at least 10⁹ or at least 10¹⁰ viral particles (vp); c) the composition does not comprise an adjuvant; d) the influenza virus HA antigen is from an influenza A virus, optionally wherein the influenza A virus is subtype H1N1 and/or subtype H3N2; e) the method induces a seroprotective immune response lasting for at least 12 months; and/or, f) the method induces a combined mucosal, humoral and T cell protective immune response provides protection against influenza A virus and influenza B virus subtype. In some embodiments, the methods disclosed herein comprise intranasally administering into the nostrils of a human subject the effective amount of the immunogenic composition, wherein: during administration the nostrils of the human subject are tilted upward; and, the effective amount induces a protective immune response against a respiratory pathogen. In some embodiments, the effective amount comprises at least about 10⁹ vp. In some embodiments, the immunogenic composition can be administered in two doses split approximately evenly between two nostrils of the human subject. In some embodiments, the human subject can be in the supine position during administration and remains in the supine position for at least about 30 minutes following administration. In some embodiments, the heterologous antigen can be an influenza antigen, optionally wherein the influenza antigen is the hemagglutinin (HA) surface antigen. In some embodiments, the heterologous antigen can be a coronavirus antigen, optionally wherein the coronavirus antigen is a SARS-Cov-2 antigen. In some embodiments, the replication deficient adenovirus vector does not contain or express a heterologous antigen. In some embodiments, the human subject can be a human being with early coronavirus infectious disease. In some embodiments, the methods disclosed herein can prevent clinical worsening in the human being, which in preferred embodiments can be defined as at least about a 4.0% decrease from baseline in resting SpO₂ as determined by pulse oximetry on two consecutive measurements following administration. In some embodiments, the clinical worsening can be determined by measuring at least one characteristic selected from the group consisting of maximal severity of COVID-19 after treatment as assessed by average decrease in resting SpO₂ from baseline resting SpO₂ at screening during the 14 days following administration, average increase in resting pulse rate from baseline resting pulse rate at screening during the 14 days following administration, proportion of patients requiring hospitalization on Day 42 following administration according to the level of oxygen supplementation required, and all-cause mortality through Day 42 following administration. In some embodiments, the protective immune response against a respiratory pathogen can be elicited within about 24 hours. In some embodiments, the protective immune response against a respiratory pathogen can be elicited within about 1 to 2 days. In some embodiments, the methods comprise instructing or otherwise keeping the mammalian subject to remain in the modified sitting, reclining, or supine position for 30 minutes after the intranasal administration.

In some embodiments, this disclosure provides method(s) for transmucosal administration of a therapeutic dose of a non-replicating viral vectored immunogenic composition to a human subject, wherein the methods comprise: administering intranasally to the mammalian subject an immunogenic composition comprising an effective amount of at least 10⁸ viral particle (vp) of replication deficient adenovirus vector, with or without containing and expressing a heterologous antigen codon optimized coding sequence for the mammalian subject, wherein the human subject during administration is in a modified sitting, reclining, supine, or other position wherein nostrils of the human subject are tilted upward; whereby the therapeutic dose administered transmucosally induces a protective immune response against a respiratory pathogen. In some embodiments, the respiratory pathogen is selected from the group consisting of a orthomyxovirus; a paramyxovirus; a rhinovirus; a coronavirus; an influenza virus, a respiratory syncytial virus (RSV); a common cold virus; and a measles virus. In some embodiments, the replication deficient adenovirus vector contains and expresses a heterologous antigen selected from an influenza virus antigen or a coronavirus antigen. In some embodiments, the heterologous antigen is a SARS-Cov-2 antigen. In some embodiments, the heterologous antigen is an influenza antigen.

In some embodiments, the mammalian subject treated by the methods and/or using the reagents (e.g., vectors) disclosed herein is a companion animal, a domesticated animal, a food-or feed-producing animal, a livestock animal, a game animal, a racing animal, or a sport animal. In some embodiments, the mammalian subject is a cow, a horse, a dog, a cat, a goat, a sheep, or a pig. In some embodiments, the mammalian subject is a human, optionally wherein the human is an adult. Other embodiments of the same are also contemplated herein, as would be understood by those of ordinary skill in the art.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1

### Preparation of Anthrax Immunogenic Composition Pharmaceutical Formulation (NasoShield)

The present adenoviral vector is an E1/E3-deleted, replication deficient (RD)-Ad5 vector that expresses the protein of interest (e.g., B. anthracis PA) within respiratory epithelial cells and/or other organs/tissues. The vector contains a genetic insert such as the transgene encoding the PA₈₃ (protective antigen—83 kDa form) gene from B anthracis (or any other gene of interest in trans). The recombinant Ad5 vector lacks the E1 region of the viral genome which renders the virus replication deficient (RD) and incapable of producing infectious virus particles upon entry into a host cell. An additional deletion of nucleotides in the E3 region of the vector removes genes that are involved in evading the host immune response and are dispensable for virus replication. An expression cassette consisting of a cytomegalovirus transcriptional enhancer/promoter to drive the expression of the PA₈₃ gene, (or other specific transgene), and a Simian Virus 40 polyadenylation signal has been inserted in place of the E1 gene sequences. FIG. 1 provides a schematic diagram of the RD-Ad5 vector and identifies those sequences from the parent adenovirus genome that are retained in the vector.

Protective antigen (PA) plays a key role in anthrax disease by binding host cells and combining with 1 of 2 other proteins to form the exotoxins responsible for cell death [Wright 2010]. Antibody against PA has been well established as critical to prevention of anthrax disease. TNA is a key biomarker and basis of the animal rule approval of currently licensed anthrax immunogenic composition (e.g., vaccine) (Moldovan I R, Tary-Lehmann M. Improving immunogenicity, efficacy and safety of vaccines through innovation in clinical assay development and trial design: the Phacilitate Vaccine Forum, Washington D.C. 2011. Hum Vaccin. 2011 June; 7(6):610-2).

The replication deficient adenoviral vector containing and expressing B. anthracis protective antigen (PA) codon optimized for a mammalian subject was prepared following procedures detailed in [Lui J. et al.; A protocol for rapid generation of recombinant adenoviruses using the AdEasy system; Nat. Protoc. (2007) 2(5):1236-47].

NasoShield was manufactured by propagation of the RD-Ad5 vector in replication-permissive PER.C6 cells, followed by purification of the virus from the infected cell harvest, and the final product included the following excipients: Tris HCl (pH 7.4), histidine, sucrose, sodium chloride, magnesium chloride, polysorbate 80, ethylenediaminetetraacetic acid, and ethanol.

For the clinical study (Example 2), NasoShield was supplied in single-use glass vials each containing a nominal volume of 0.7 mL of a sterile, frozen suspension of immunogenic composition (e.g., vaccine) formulated to deliver the nominal dose of 1×10⁹, 1×10¹⁰, or 1×10¹¹ viral particles (vp).

For the animal study (Example 3), NasoShield (also referred to as AdVAV in earlier studies) was supplied in single-use glass vials each containing a nominal volume of 0.7 mL of a sterile frozen suspension of immunogenic composition (e.g., vaccine) containing 1.5×10⁹ vp (batch number: 17255-001).

### Example 2

### First-In-Man Phase 1 Study to Evaluate Safety and Immunogenicity of NasoShield

A first-in-man Phase 1 study was conducted to evaluate safety and immunogenicity of NasoShield. An objective of the study was to evaluate the safety and the immunogenicity of NasoShield associated with the anti-protective antigen (PA)-specific humoral immune response to NasoShield administered by intranasal spray as a single dose of 1×10⁸, 1×10⁹, 1×10¹⁰, or 1×10¹¹ vp and as 2 doses (3 weeks apart) of the highest well tolerated of these doses (e.g. 1×10¹¹ vp), wherein the subjects were in a normal sitting position (i.e., head facing directly forward with eyes focused straight ahead on an object at eye level and nostrils angled downward) when NasoShield was administered.

NasoShield was supplied in single-use glass vials, each containing a nominal volume of 0.7 mL of a sterile, frozen suspension of immunogenic composition (e.g., vaccine) formulated to deliver the nominal doses of 1×10⁹, 1×10¹⁰, or 1×10¹¹ viral particles (vp). The lowest dose (1×10⁸ vp) was compounded at the site pharmacy by diluting the 1×10⁹ vp vial with appropriate quantity of diluent buffer in a controlled aseptic area. A 0.5 mL dose was split between the two nostrils (“divided dose”) and administered as an intranasal spray using a 1 mL tuberculin syringe fitted with a Teleflex LMA™ MAD® Nasal Intranasal Mucosal Atomization Device (LMA MAD) on Day 1 (single-dose cohorts; Part A of the trial) or on Days 1 and 22 (2-dose cohort; Part B of the trial). The population studied was healthy, immunocompetent adults between the ages of 18 and 49 years. The subjects of this clinical trial were administered NasoShield positioned in a normal sitting position.

In Part A of this clinical trial, 120 subjects were enrolled into 4 sequential cohorts of 30 subjects each defined by the NasoShield dose (1×10⁸, 1×10⁹, 1×10¹⁰, and 1×10¹¹ vp). Within each cohort (and the sentinel group in the first dose cohort), subjects were randomized in a 4:1:1 ratio to receive 1 intranasal dose of NasoShield (Day 1), one intranasal dose (i.e., intranasal spray) of placebo (Day 1). In Part B of this clinical trial, 25 subjects were randomized in a 4:1 ratio to receive two intranasal doses (i.e., by intranasal spray) of NasoShield at the highest well-tolerated dose from Part A (1×10¹¹ vp) or placebo 21 days apart (Days 1 and 22).

A planned interim analysis was conducted when all data through the Day 57 visit in Part B (35 days after Dose 2) were available, monitored for all subjects, and confirmed by the Investigator for all subjects. The reporting periods for reactogenicity events and AEs other than SAEs, medically attended AEs (MAEs), and new-onset chronic illnesses (NCIs) was completed for all subjects at the time of the interim analysis. There was no statistically significant difference between the overall NasoShield group and the placebo group in the incidence of local reactogenicity events (P=0.439) or systemic reactogenicity events (P=0.573) after any dose. The Ad5 vector was cleared from the nasopharynx in most subjects who received NasoShield in Part A (92.0%) by Day 8 and in all subjects by Day 15 (note: Part B samples were not tested). The number of subjects with any shedding and magnitude of shedding showed a dose-dependent pattern at Days 4 and 8.

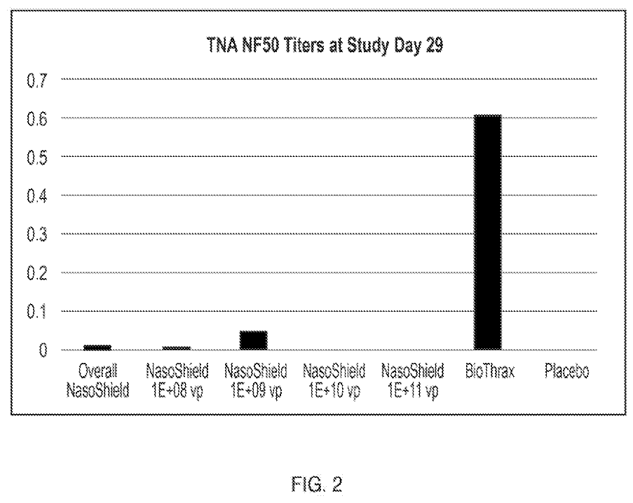

In Part A, only a few subjects in the NasoShield groups met the definition of responder (≥4-fold rise at 28 days post dose) for IgG or TNA. In Part B, 10 subjects (66.7%) in the NasoShield 1×10¹¹ vp group met the definition of IgG response after Dose 1 and 9 subjects (60.0%) after Dose 2; however, those meeting the definition of responder had very low GMTs. In both Part A and Part B, only a few subjects in the NasoShield groups met the definition of responder for TNA NF₅₀ or ED₅₀. At Day 29, NasoShield at any dose elicited an IgG response in only 21.0% of subjects and a TNA response as measured by NF50 and ED50 in 3.7% and in 5.4% of subjects, respectively. Thus, immunogenicity analyses showed minimal immunoglobulin G (IgG) response elicited by NasoShield at 28 days after one dose (Day 29) or two doses (Day 50). No toxin neutralizing antibody (TNA) response was elicited in any subject among the 1×10¹⁰ or 1×10¹¹ dose groups, and in very few of the subjects in the lower dose groups. See FIG. 2. The limited response to NasoShield made it difficult to assess the effect of pre-dose Ad5 serum antibody levels on immune response. It is again noted that in this clinical trial, NasoShield was administered to subjects positioned in the normal sitting position (i.e., the nostrils were not tilted back or in the supine position). These findings were unexpected and inconsistent with previous animal studies and the clinical trial of NasoVAX, an intranasal influenza vaccine based on the same RD-Ad5 vector and using the same administration device (LMA MAD Nasal Intranasal Mucosal Atomization Device) and procedures.

### Example 3

### Immunogenicity Differences for Positional Administration of NasoShield in New Zealand White (NZW) Rabbits

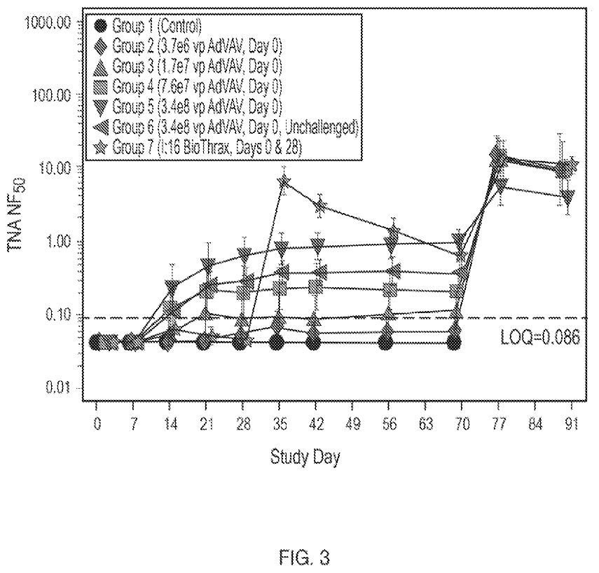

The immunogenicity data from the clinical trial (Example 2) were in contrast to the high levels of toxin neutralizing antibody (TNA) response seen in pre-clinical studies, even at very low doses of NasoShield. See FIG. 3. An investigation was initiated to find potential root cause(s) that may have led to this outcome in the clinical study. As part of the investigation, product potency, formulation components, contaminant profile as well as other key parameters were assessed. The delivery device, the mode of intranasal administration, and position of administration were studied in a rabbit study to delineate differences if any, in immunogenicity generated by NasoShield, that could be related to or could be dependent on any of the above indicated parameters.

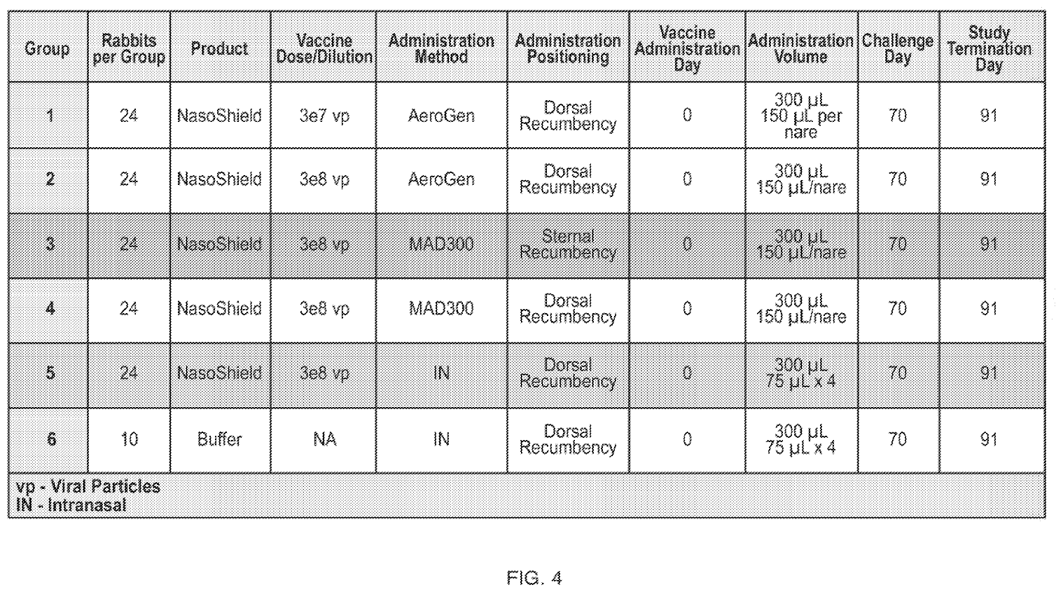

FIG. 4 provides a summary of the study design. The rabbits were vaccinated with NasoShield in sternal (nostrils facing down in a normal resting position, corresponding to the normal sitting position in the human clinical trial of Example 2) and dorsal (rabbits on their back with nostrils facing up; corresponding to the supine position of the human clinical trial of Example 5 and 6) positions using the intranasal delivery device used in the clinical trial of Example 2. The comparator arm was instillation (droplets) of NasoShield administered in the dorsal position. This type of administration was used for the pre-clinical studies prior to commencement of the clinical trial. The NasoShield viral particles (vp) were administered, in the liquid form by or using a nebulizer or atomizer (AeroGen or LMA MAD/MAD300), with the rabbit positioned in the dorsal or sternal position. The number of particles administered was 3×10⁸ vp varying the device and position with a control group treated with buffer only and a lower vp count comparative group treated with 3×10⁷ vp using the AeroGen nebulizer. The sternal position in rabbits most closely mimics the human normal sitting posture (with nostrils facing downward or slightly inclined) which was implemented for the administration of NasoShield in the clinical trial.

NasoShield immunogenicity was assessed via two assays: the anti-PA IgG Enzyme Linked Immunosorbent Assay (ELISA) which measures PA-specific IgG levels, and the toxin neutralization assay (TNA), a functional assay that measures and quantifies the ability of serum to neutralize B. anthracis lethal factor (LT). The current study was designed to compare the immunogenicity of NasoShield when administered to NZW rabbits via intranasal liquid instillation, atomization with LMA MAD®, or nebulization with an intranasal nebulizer designed for immunogenic composition (e.g., vaccine) administration, but with different positions to account for sternal and dorsal administration, and subsequently, the efficacy of the administered doses as measured by survival following a lethal inhalation B. anthracis Ames spore challenge.

Toxin neutralization assays (TNA) assess the ability of antibodies within sample sera to protect cells in culture from toxin. In one embodiment the toxin is B. anthracis lethal factor (LT). The cells are exposed to sample sera, usually from an immunized animal, which is diluted down the plate. Toxin is then added, and the cells incubated. Any live cells can be visualized using reagents such as diphenyltetrasolium bromide (MTT) which will be metabolized by live cells to give a vivid color (for MTT, the resulting color is purple formazan crystals). The further toward death the cells are, the less color will be developed (i.e., be detected). Completely dead cells will give no color. In the presence of any antibody towards the toxin, the antibody should bind the toxin will therefore help reduce the effect of the toxin on the cells and should prevent them from dying off. The plates the cells have grown on can then be analyzed on a plate reader to assess the absorbance from each well and therefore the concentration at which the toxin can kill half the cells

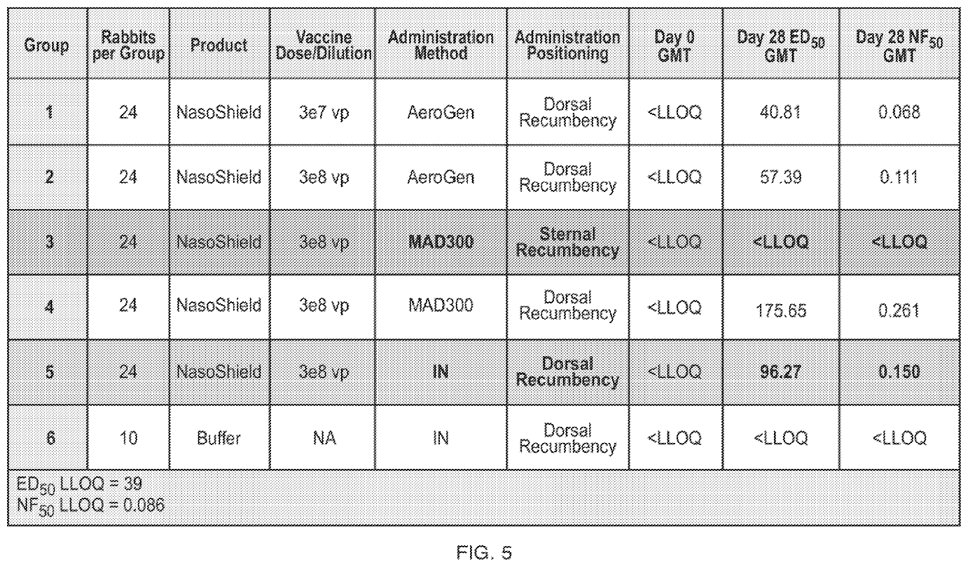

The TNA ED₅₀ and TNA NF₅₀ from vaccinated rabbits were evaluated in a validated TNA assay at day 28 post vaccination. See FIG. 5.

The data indicated that both the LMA MAD as well as instillation were able to generate a TNA response. However, only the dorsal administration using LMA MAD (MAD300) or the AeroGen model nebulizers was able to generate an immune response. See FIG. 4.

In all 24 animals tested, the sternal (i.e., non-supine; normal sitting position) administration of the immunogenic composition (e.g., vaccine) generated no immunogenic response in the TNA assay. Additionally, even a 10-fold lower dose using another device (Aerogen) but administered in the dorsal (i.e., supine) position generated a dose appropriate immune response. This indicates that the position of administration (e.g., the position of the head/nostrils during intranasal administration) is a critical parameter for generating an immunogenic response for intranasally administered immunogenic composition (e.g., vaccine) and potentially other therapeutics. The data shows that tilting the head (i.e., nostrils angled upward or above the head, the dorsal or supine position) may allow for therapeutic agent/immunogenic composition (e.g., vaccine) to access and/or localize in specific areas of the nasal passages, sinuses and adjacent tissues/organs, which may be critical for eliciting an immune response. The tilting of the head back with nostrils and chin lifted upward is therefore the preferred position for administration of NasoShield. The areas accessed by intranasal administration in sternal/normal sitting posture (i.e., with nostrils not facing upwards) may be different and/or sub-optimal for generating a strong immune response.

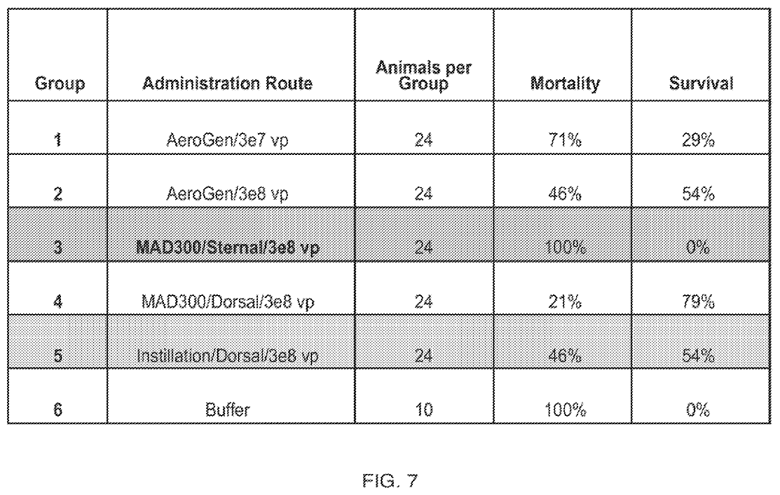

Survival rate data of the rabbits' post NasoShield immunization and spore challenge is shown in FIG. 7. The groups of rabbits, 24 per group, were treated with varying number of viral particles using the AeroGen nebulizer (groups 1 and 2) in the dorsal position, the MAD300 nebulizer in the sternal and dorsal position respectively (groups 3 and 4), without a device in the dorsal position, i.e., instillation (group 5), and as a control the rabbits were treated with only a buffer solution by instillation (group 6). The lowest mortality of 21% (79% survival) was observed for the rabbits with the immunogenic composition (e.g., vaccine) administered using a MAD300 atomizer and the rabbit positioned in the dorsal position (i.e., it acted as a vaccine). Administration using the MAD 300 in the sternal position provided no protection against anthrax with a 100% mortality rate (zero survival), similar to the buffer control (i.e., neither acted as a vaccine). The AeroGen nebulizer in dorsal position was not as effective as the MAD300 atomizer in the dorsal position, with a higher mortality rate of 46% at comparable virus particle count and an even higher mortality rate, 71%, when the vp count was lowered to 3×10⁷. However, it still provided some measure of protection from anthrax challenge better than the sternal position of administration using MAD300.

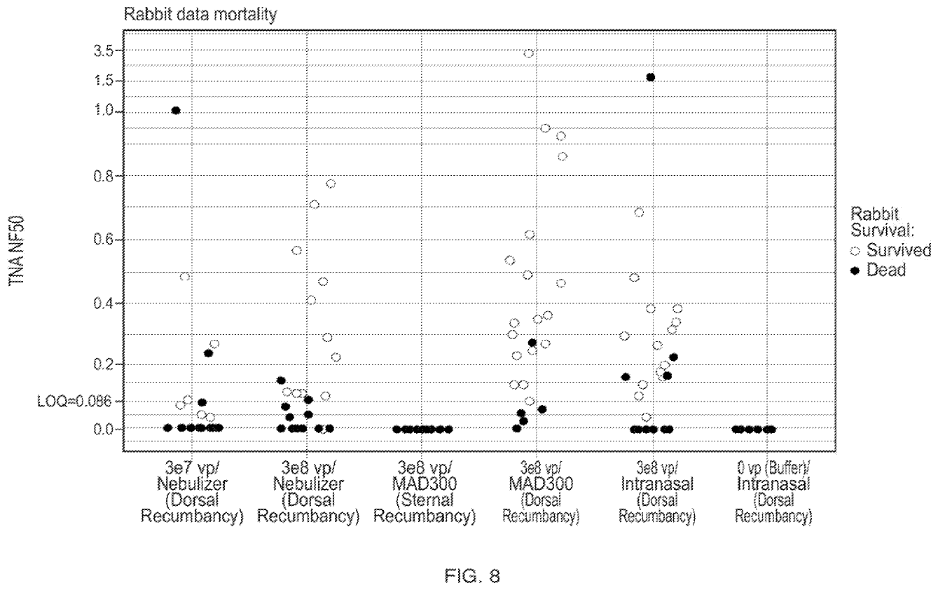

A comparison of the TNA results to the Rabbit mortality data for each group are presented in FIG. 8, wherein a TNA response generally corresponds to survival of the rabbits vaccinated with NasoShield and challenged with Anthrax spores.

The position of the subject during the administration of immunogenic agents, such that nostrils are tilted upwards or elevated about the head of the subject, while in a modified sitting, reclining and/or supine posture, is correlated with the generation of a strong immunogenic response via intranasal administration of an immunogenic composition (e.g., vaccine)/therapeutic agent in both animals and humans. As shown herein, when administered to the subject in a dorsal position, the immunogenic composition can act as a vaccine.

### Example 4

### Immunogenicity Differences for Positional Administration of NasoVAX in New Zealand White (NZW) Rabbits

A. Preparation of Monovalent Influenza Pharmaceutical Formulation (NasoVAX)

The replication-deficient adenoviral vector containing and expressing influenza virus hemagglutinin antigen codon optimized for the human subject was prepared following procedure detailed in Lui J. et al. (A protocol for rapid generation of recombinant adenoviruses using the AdEasy system; Nat. Protoc. (2007) 2(5):1236-47). The adenoviral vector used in this study (NasoVAX) is an E1/E3-deleted, replication deficient (RD)-Ad5 vector that expresses the protein of interest (e.g., influenza HA) within respiratory epithelial cells. In the case of NasoVAX, the vector contains a genetic insert encoding the HA surface protein antigen from influenza type A or B. The recombinant Ad5 vector lacks the E1 region of the viral genome (nucleotides 343 to 3511), which renders the virus replication defective (RD) and incapable of producing infectious virus particles upon entry into a host cell. An additional deletion of nucleotides in the E3 region of the vector removes genes that are involved in evading the host immune response and are dispensable for virus replication. An expression cassette consisting of a cytomegalovirus transcriptional enhancer/promoter to drive the expression of the HA gene, a bioengineered HA gene, and a Simian Virus 40 polyadenylation signal has been inserted in place of the E1 gene sequences. FIG. 1 provides a schematic diagram of the RD-Ad5 vector and identifies those sequences from the parent adenovirus genome that are retained in the vector. For this study, the RD-Ad5 vector contained a genetic insert encoding the hemagglutinin (HA) surface protein antigen from an A/California/04/2009(H1N1)-like strain of influenza (AdcoCA09.HA). NasoVAX was manufactured by propagation of this RD-Ad5 vector in replication-permissive PER.C6 cells, followed by purification of the virus from the infected cell harvest, and the final product included the following excipients: Tris HCl (pH 7.4), histidine, sucrose, sodium chloride, magnesium chloride, polysorbate 80, ethylenediaminetetraacetic acid, and ethanol. NasoVAX was provided in single-use glass vials each containing a nominal volume of 0.7 mL of a sterile, frozen suspension of immunogenic composition (e.g., vaccine) formulated as 2×10¹⁰ vp/mL in A195 buffer (batch number: 17143001), and used in the studies below.

B. Immunization of New Zealand White (NZW) Rabbits Using NasoVAX

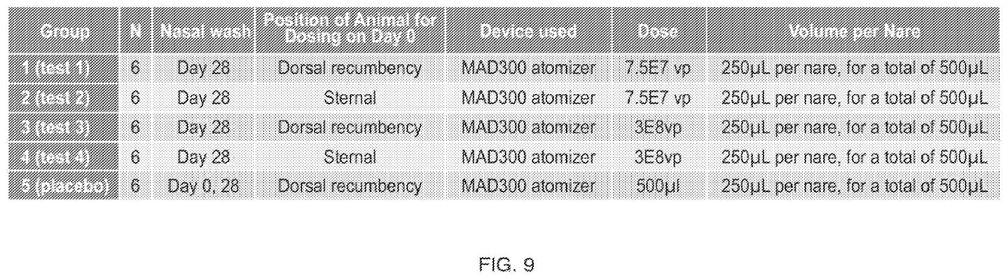

The NasoVAX viral particles (vp) were administered intranasally to rabbits in the liquid form using the LMA MAD/MAD300 atomizer (described above) or AeroGen nebulizer, each rabbit being positioned in the dorsal (e.g. supine) or sternal (e.g. normal sitting) position. In the dorsal position, the rabbits were positioned on their back with nostrils facing up while in the sternal position the nostrils were facing down in a normal resting position corresponding to the human normal sitting position. The number of viral particles (vp) administered using the LMA MAD/MAD300 device was 7.5E7 or 3E8 vp, varying the administration position as indicate below (dorsal or sternal). Along with the LMA MAD/MAD300 was a control group treated with buffer only (“A195 buffer”). A comparator arm to which instillation (droplets) of NasoVAX was administered in the dorsal position was also included. FIG. 9 shows the Study Groups (Groups 1-5).

In the case of influenza immunogenic composition (e.g., vaccine), antibodies measured in a hemagglutination inhibition assay (HAI) is a surrogate of protection, wherein the HAI is based on the ability of antibodies, if present in the serum, to prevent agglutination between erythrocytes and viral hemagglutinin. In these studies, NasoVAX immunogenicity was assessed using HAI. Briefly, the serum of test rabbits to which NasoVAX was administered were tested by HAI for the presence of antibodies against the influenza virus and compared to that of control rabbits to which NasoVAX was not administered, the rabbits being in the dorsal or sternal position upon administration as indicated. The results are shown in Tables 1 and 2 below (the HAI at day zero being zero), as well as FIGS. 10-13.

TABLE 1
Day 14 HAI Results

| Group | Position | Dose (vp) | Animal # | HAI |
|---|---|---|---|---|
| 1 | Dorsal | 7.5 × 10⁷ | 1 | 10 |
| | Recumbency | | | |
| | | | 2 | 10 |
| | | | 3 | 5 |
| | | | 4 | 20 |
| | | | 5 | 10 |
| | | | 6 | 5 |
| 2 | Sternal | 7.5 × 10⁷ | 7 | 5 |
| | | | 8 | 5 |
| | | | 9 | 10 |
| | | | 10 | 5 |
| | | | 11 | 5 |
| | | | 12 | 5 |
| 3 | Dorsal | 3 × 10⁸ | 13 | 10 |
| | Recumbency | | | |
| | | | 14 | 80 |
| | | | 15 | 80 |
| | | | 16 | 5 |
| | | | 17 | 40 |
| | | | 18 | 20 |
| 4 | Sternal | 3 × 10⁸ | 19 | 5 |
| | | | 20 | 5 |
| | | | 21 | 5 |
| | | | 22 | 5 |
| | | | 23 | 10 |
| | | | 24 | 5 |
| 5 | Dorsal | A195 | 25 | 5 |
| | Recumbency | buffer | | |
| | | | 26 | 5 |
| | | | 27 | 5 |
| | | | 28 | 5 |
| | | | 29 | 5 |
| | | | 30 | 5 |

TABLE 2
Day 28 HAI Results

| Group | Position | Dose (vp) | Animal # | HAI |
|---|---|---|---|---|
| 1 | Dorsal | 7.5 × 10⁷ | 1 | 5 |
| | | | 2 | 10 |
| | | | 3 | 5 |
| | | | 4 | 60 |
| | | | 5 | 20 |
| | | | 6 | 5 |
| 2 | Sternal | 7.5 × 10⁷ | 7 | 5 |
| | | | 8 | 5 |
| | | | 9 | 5 |
| | | | 10 | 5 |
| | | | 11 | 5 |
| | | | 12 | 5 |
| 3 | Dorsal | 3 × 10⁸ | 13 | 80 |
| | | | 14 | 40 |
| | | | 15 | 40 |
| | | | 16 | 160 |
| | | | 17 | 40 |
| | | | 18 | 160 |
| 4 | Sternal | 3 × 10⁸ | 19 | 5 |
| | | | 20 | 5 |
| | | | 21 | 5 |
| | | | 22 | 5 |
| | | | 23 | 5 |
| | | | 24 | 5 |
| 5 | Dorsal | A195 | 25 | 5 |
| | recumbency | buffer | | |
| | | | 26 | 5 |
| | | | 27 | 5 |
| | | | 28 | 5 |
| | | | 29 | 5 |
| | | | 30 | 5 |

TABLE 3
Geometric Mean Titer (GEOMEAN) and GMR of HAI data

| | Administration | GEOMEAN | GMR |
|---|---|---|---|
| Day 0 | Dorsal 7.5 × 10⁷ vp | 6.3 | — |
| | Sternal 7.5 × 10⁷ vp | 5 | — |
| | Dorsal 3 × 10⁸ vp | 5 | — |
| | Sternal 3 × 10⁸ vp | 5 | — |
| | Dorsal Placebo | 5 | — |
| Day 14 | Dorsal 7.5 × 10⁷ vp | 8.9 | 1.4 |
| | Sternal 7.5 × 10⁷ vp | 5.6 | 1 |
| | Dorsal 3 × 10⁸ vp | 25 | 5.0 |
| | Sternal 3 × 10⁸ vp | 5.6 | 1 |
| | Dorsal Placebo | 5 | 1 |
| Day 28 | Dorsal 7.5 × 10⁷ vp | 10.7 | 1.7 |
| | Sternal 7.5 × 10⁷ vp | 5 | 1 |
| | Dorsal 3 × 10⁸ vp | 71 | 14 |
| | Sternal 3 × 10⁸ vp | 5 | 1 |
| | Dorsal Placebo | 5 | 1 |

The data presented in Tables 1-3 are also presented in FIGS. 10-13. As shown in FIGS. 10-13, the “Dorsal 3×10⁸ vp” group to which 3×10⁸ vp administered using the LMA MAD/MAD300 device to rabbits positioned in the dorsal position showed that those rabbits were producing anti-influenza virus antibodies by day 14 and that the amount increased by day 28. These HIA assays did not indicate any detectable anti-influenza antibodies were produced by animals administered the lower dose (7.5×10⁷) in either the dorsal or sternal position, or by animals administered the higher dose (3×10⁸) in the sternal position. Thus, this study surprisingly shows that the position of the subject during the administration of immunogenic compositions disclosed herein, such that nostrils are tilted upwards (i.e., the dorsal position), is correlated with the generation of a strong immunogenic response via intranasal administration of an immunogenic composition (e.g., vaccine)/therapeutic.

### Example 5

### Double-Blind, Randomized, Placebo-Controlled, Study of the Safety and Immunogenicity of NasoShield Administered as One or Two Doses in Different Dosing Positions

This study is a Phase 1b, randomized, double-blind, placebo-controlled clinical trial to evaluate the safety and immunogenicity of NasoShield in healthy adults 18 to 49 years of age. Subjects will be screened within 28 days before randomization (Day 1). Approximately 42 subjects who meet all inclusion and no exclusion criteria and provide written informed consent will be enrolled and randomized into three (3) parallel groups defined by dosing position (normal sitting, sitting with nostrils elevated above head, or supine) and number of doses (single dose on Day 1 or 2 doses on Days 1 and 29) to receive 0.5 mL intranasal doses of NasoShield (1×10¹¹ vp) or saline placebo. Within each of the following three groups, subjects will be randomized in a blinded fashion in a 5:2 ratio to receive NasoShield 1×10¹¹ vp or saline placebo given as an intranasal spray: Group 1: NasoShield 1×10¹¹ vp or saline placebo on Day 1 and saline placebo on Day 29 in normal sitting position (no dose holding time); Group 2: NasoShield 1×10¹¹ vp or saline placebo on Day 1 and Day 29 in modified sitting position with nostrils elevated above head (i.e., nostrils tilted upwards) for 3 minutes followed by a normal sitting position for 27 minutes; and, Group 3: NasoShield 1×10¹¹ vp or saline placebo on Day 1 and Day 29 in supine position (on back with nostrils pointing up) for 30 minutes. Subjects in Group 1 who receive NasoShield on Day 1 will receive placebo on Day 29, and subjects who receive placebo on Day 1 will receive placebo on Day 29 as well. Subjects in Groups 2 and 3 who receive NasoShield on Day 1 will receive NasoShield on Day 29, and subjects who received saline placebo on Day 1 will receive saline placebo on Day 29. The subject is instructed not to blow his/her nose for 30 minutes after dosing.

Group 1: Normal Sitting Position. Place subject in a chair, upright position with feet flat on the floor (Photo 1 below). Instruct subject to keep head facing directly forward with eyes focused straight ahead on an object at their eye level. Instruct subject to hold this position, i.e., do not tilt head back or forward during the dosing. Once dosing administration is complete, there is no additional hold time required. Subject should be instructed to avoid blowing nose for 30 minutes.

Group 2: Sitting with Nostrils Elevated Above Head. Place subject in a chair that will allow subject to fully extend neck/tilt head back, i.e., a chair with a low back. Instruct subject to tilt head back/lift chin as far as comfortably possible with eyes focused on the ceiling. Instruct subject to hold this position, i.e., do not lean forward/drop chin or move head during the dosing or for three minutes post dosing. If needed, to maintain the position for three minutes, the subject may hold a towel behind neck for support. After three minutes, the subject should return to a normal sitting position and maintain for 27 minutes. Subject should be instructed to avoid blowing nose for 30 minutes.

Group 3: Supine Position. Place subject in the supine position (laying on their back), flat without a pillow. Instruct subject to keep head facing forward with eyes focused straight ahead on the ceiling directly above. Instruct subjects to hold this position, i.e., do not sit up or turn head during the dosing or for thirty minutes post dosing.

The primary objective of this study is to evaluate the safety of NasoShield administered as an intranasal spray of 1×10¹¹ viral particles (vp) as a function of number of doses and dosing position. This primary objective will be measured by determining: 1) Reactogenicity: counts and percentages of subjects with local events: nasal irritation, sneezing, nasal congestion, epistaxis, sore throat, change in smell, change in taste, change in vision, eye pain for 7 days after vaccination; and, 2) Adverse events (AEs): counts and percentages of subjects with AEs from Day 1 to Day 57; medically attended AEs (MAAEs), serious AEs (SAES), and new-onset chronic illnesses (NCIs) from Day 1 to Day 210.

A first secondary objective of this study is to evaluate the humoral immunogenicity of NasoShield administered as an intranasal spray of 1×10¹¹ vp as a function of number of doses and dosing position. This first secondary objective is ascertained by determining: 1) anti-protective antigen (PA) immunoglobulin G (IgG) titer measured by enzyme-linked immunosorbent assay (ELISA) in serum by determining the geometric mean titer (GMT) (the antilog of the mean of the log-transformed titers), the geometric mean ratio (GMR): the ratio of postvaccination and prevaccination GMTs within the same dose group, and responder rate; and, 2) the 50% neutralization factor (NF50) and 50% effective dilution (ED50) titers measured by toxin neutralization assay (TNA) in serum (GMT, GMR, and responder rate).

A second secondary objective of this study is to evaluate the mucosal immune response to NasoShield administered as an intranasal spray of 1×10¹¹ vp as a function of number of doses and dosing position. This second secondary objective is ascertained by determining anti-PA immunoglobulin A (IgA) titer measured by ELISA (GMT GMR).

A third secondary objective of this study is to evaluate the effect of pre-dose adenovirus serotype 5 (Ad5) serum antibody levels on the immunogenicity of NasoShield as a function of number of doses and dosing position. This third secondary objective is ascertained by determining anti-PA IgG, TNA NF50, and anti-PA IgA GMTs and GMRs based on pre-dose Ad5 antibody levels measured by microneutralization in serum.

A fourth secondary objective of this study is to measure induction of anti-Ad5 immunity as a function of number of doses and dosing position. This fourth secondary objective is ascertained by determining Ad5 antibody GMT measured by microneutralization in serum.

Subjects undergo targeted and symptom-driven physical examination at various times throughout the trial (e.g., to study any adverse effects (AEs)). Serum samples for immunogenicity testing (measurement of anti-PA IgG titer by ELISA and measurement of NF50 and ED50 titer by TNA) are obtained at Days 15, 29, 57, 91, and 210. An additional serum sample is taken on Days 29 and 57 for measurement of Ad5 titer by microneutralization assay. Nasopharyngeal swabs for detection of replication-deficient (RD)-Ad5 vector shedding by quantitative polymerase chain reaction (qPCR) assay are obtained on Days 4, 8, 15, 36, and 57 and for anti-PA IgA by ELISA on Days 15, 36, and 57. Swabs are not collected in the 14 days prior to the administration of the NasoShield/placebo dose at the Day 1 or Day 29 visits. Two interim analyses of immunogenicity and safety data (including summaries of AE, reactogenicity, concomitant medications and vaccines, vital signs, ECG, and laboratory data and listing of any viral culture results) are conducted: the first when all data through the Day 29 visit are available for all subjects, and the second when all data through the Day 57 visit are available for all subjects.

The sample size for this study is selected as adequate and reasonable for a review of the safety and immunogenicity profile of NasoShield administered by intranasal spray given in different dosing positions, rather than for statistical power. The sample size permits initial estimates of reactogenicity. Given a total of 30 subjects receiving NasoShield, the study has an 80% probability of detecting at least 1 AE which occurs at a rate of 5.2%. If no SAEs are observed among the 30 subjects who receive NasoShield, an approximation to the 1-sided upper bound of the 95% confidence interval (CI) on the rate of SAE occurrence would be 11.6%. The following analysis populations is used: 1) Safety Population: All subjects who provide informed consent, are randomized, and receive at least 1 dose of investigational product (IP); 2) Evaluable Population: All subjects in the Safety Population who have anti-PA IgG results on Day 1 and Day 29; and, 3) Per-protocol (PP) Population: All subjects in the Safety Population that received the assigned dose of the test article in accordance with the protocol; will have anti-PA IgG results at Days 1, 15, and 29; and will have no major protocol deviations affecting the primary immunogenicity outcomes as determined by the Sponsor prior to database lock. The primary immunology analyses will be conducted using the Evaluable Population, with a sensitivity analysis based on the PP Population. No imputation for missing data will be performed. Data will be transformed as appropriate prior to analysis. Baseline is defined as the sample collected prior to IP administration on Day 1. The primary variables of interest for assessment of humoral immune response to NasoShield are anti-PA IgG and TNA NF₅₀ and ED₅₀ titers. The following measures and their 95% CIs are summarized by group: GMT at baseline and postvaccination on Days 15, 29, 57, 91, and 210; GMR on Days 15, 29, 57, 91, and 210; and, Responder rate on Days 15, 29, 57, 91, and 210. Mucosal responses (anti-PA IgA) are summarized by geometric mean at baseline and postvaccination on Days 15, 36, and 57. For the effect of pre-dose Ad5 serum antibody levels on immunogenicity of NasoShield on Days 29 and 57, analysis is performed using analysis of covariance (ANCOVA), with baseline Ad5 titer as a covariate. Safety analyses is performed using the Safety Population. The number (percentage, 95% CI) of subjects with local events and systemic events is summarized by group. Reactogenicity events are summarized by severity. The number (percentage, 95% CI) of subjects with AEs from Day 1 to Day 57 (including MAAEs, NCIs, SAEs) are summarized for each Medical Dictionary for Regulatory Activities (MedDRA) system organ class and preferred term and by group. The number (percentage) of subjects with MAAEs, with NCIs, and with SAEs from Day 1 to Day 210 are summarized in a similar fashion. The number (percentage, 95% CI) of subjects with AEs by severity and by relationship to IP are also summarized. Summary statistics for continuous parameters (safety laboratory tests and vital signs) are presented by group as follows: prevaccination, postvaccination, and change from prevaccination to postvaccination assessment. The number and percentage of subjects with postvaccination safety laboratory values or vital sign values recorded as newly abnormal (i.e., an event with an increase in the toxicity grade relative to the baseline value and with a severity grade of moderate or higher) after study vaccination are tabulated. Shift tables which cross-tabulate the prevaccination and postvaccination safety laboratory values of each subject by severity grade are prepared. Summaries of the number and percentage of subjects with normal, abnormal not clinically significant, and abnormal clinically significant ECG interpretations are presented. For shedding of the RD-Ad5 vector, data is summarized by count and percent positive by time point, along with median copy number. The median duration of Ad5 shedding, interquartile range, minimum and maximum duration of Ad5 shedding is determined for each NasoShield group and all NasoShield dose groups combined.

The trial endpoints may include safety information, e.g., that NasoShield is safe for administration to humans in any of the positions tested here (e.g., the normal sitting, odified sitting and dorsal/supine position), and may demonstrate that administration of NasoShield in the modified sitting position (Group 2) results in anti-PA humoral immunogenicity (per the first secondary objective) and anti-PA mucosal immune response (per the second secondary objective), and that those measures are superior in Group 2 as compared to Groups 1 (normal sitting position) or 3 (supine position). Predose Ad5 serum antibody levels on the immunogenicity of NasoShield may be found acceptable (per the third secondary objective), meaning that it does not significantly interfere with the development of an anti-PA immune response induced by NasoShield, especially in Group 2. This trial may also show that the induction of anti-Ad5 immunity induced by NasoShield, if any, is acceptable (per the fourth secondary objective), meaning that it does not significantly interfere with the development of an anti-PA immune response induced by NasoShield, especially in Group 2.

### Example 6

### NasoVAX in the Prevention of Clinical Worsening in Patients with Early Coronavirus Infectious Disease 2019 (COVID-19)

This example provides a clinical trial design for assessing the effectiveness of a replication deficient adenoviral vector (with (e.g., NasoVAX) or without (e.g., AdE) expressing a transgene) in preventing clinical worsening in patients with early COVID-19 (the disease caused by SARS-CoV-2) as a primary efficacy objective. Secondary efficacy objectives of this study include assessing the effects of NasoVAX on the severity of COVID-19, as indicated by changes in resting peripheral oxygen pulse saturation (SpO₂) and resting pulse rate and/or the effects of NasoVAX on rates of hospital admission, oxygen supplementation and mechanical ventilation.

The primary efficacy endpoint can include the proportion of patients with “clinical worsening”, which is defined herein as at least about a 4.0% decrease from baseline in resting SpO₂ by mobile pulse oximetry on two consecutive measurements during home follow-up, or hospitalization. Secondary efficacy endpoints can be determined by measuring for example and without limitation: 1) maximal severity of COVID-19 after treatment, as assessed by the following measurements: average decrease in resting SpO₂ from baseline resting SpO₂ at screening during the 14 days of home follow-up or hospitalization, average increase in resting pulse rate from baseline resting pulse rate at screening during the 14 days of home follow-up or hospitalization, proportion of patients requiring hospitalization on Day 42 according to level of oxygen supplementation required (no oxygen supplementation, oxygen supplementation with nasal cannulae, oxygen supplementation with high flow device or non-invasive ventilation, and/or mechanical ventilation); and/or 2) all-cause mortality through Day 42.

A primary safety objective of this study is to assess the safety and tolerability of NasoVAX in patients with early COVID-19. Safety endpoints can include but are not limited to incidence and severity of adverse events, oral temperatures, use of antipyretics and bronchodilators, hospital length of stay and/or intensive care unit (ICU) length of stay.

Approximately 96 patients with early COVID-19 are enrolled and randomized 1:1 to NasoVAX or placebo in three (3) successive cohorts defined by age and risk factors for severe COVID-19. Cohort 1 includes persons aged 35-49 years and the risk factors are disallowed. Cohort 2 includes persons aged 35 and above and the risk factors are disallowed. Cohort 3 includes persons aged 35 and above, with risk factors allowed. Each cohort includes approximately 20, 28, and 48 patients, respectively, with patients randomized 1:1 to the NasoVAX group or placebo within each cohort. Cohort 2 is stratified by age groups of 35-64 years and 65 years and above, whereas Cohort 3 is stratified by age groups (ages 35-64 years, ages 65 and above) and co-morbidity (one or more risk factors present, no risk factors present). This trial does not include patients meeting any one or more of the following criteria (which may be risk factors) identified during an initial Screening step: pregnant or lactating women or planning to conceive a child during the next three (3) months; resting respiratory rate>20 breaths/min on room air or resting pulse rate≥125 beats per minute; a rapidly worsening course that in the opinion of a treating medical practitioner would lead to hospitalization within the next 24-48 hours; any chronic pulmonary disease, including chronic obstructive pulmonary disease and asthma, or other respiratory diseases that could exacerbate independent of COVID-19; severe obesity, defined as body mass index≥40 kg/m²; history of severe cardiovascular disease, including but not limited to congestive heart failure, coronary artery disease, congenital heart disease, cardiomyopathies, or pulmonary hypertension, diabetes mellitus, chronic or current vaping or cigarette smoking, chronic kidney disease requiring dialysis, chronic liver disease, including but not limited to chronic viral hepatitis, non-alcoholic steatohepatitis, or cirrhosis of any cause, hemoglobin disorder, including sickle cell disease and thalassemia; history of Bell's Palsy; nasal conditions that might affect the suitability of intranasal medication, such as a history of chronic rhinitis, nasal septal defect, cleft palate, nasal polyps, or nasal surgery other than cosmetic rhinoplasty; use of hydroxychloroquine within the past 4 months, chloroquine with the past nine (9) months, or other investigational agents for COVID-19 within the past 30 days; history of conditions associated with immunocompromise, including but not limited to poorly controlled HIV, or treatments known to affect the immune system, including but not limited to oral or intravenous corticosteroids, alkylating drugs, antimetabolites, cytotoxic drugs, radiation, immune-modulating biologics (including IL-6, IL-12, Janus kinase inhibitors or antagonists), and cancer treatments, within 30 days of Screening or anticipated use within 6 months following participation in this study; and/or any medical, psychiatric, or social condition or occupational or other responsibility that in the judgment of the Investigator would interfere with or serve as a contraindication to protocol adherence, assessment of safety (including reactogenicity), or a patient's ability to give informed consent; hydroxychloroquine treatment within 4 months or chloroquine within 9 months of Screening; any treatment known to affect the immune system, including but not limited to oral or intravenous corticosteroids, alkylating drugs, antimetabolites, cytotoxic drugs, radiation, immune-modulating biologics, within 30 days of Screening; live vaccines (such as live influenza vaccinations or live travel vaccinations) within 30 days of Screening and through Day 14 post study drug administration; and/or receipt of any investigational drug or treatment within 30 days of Screening. If a patient is hospitalized due to COVID-19, there are no restrictions regarding concomitant medications. Each patient participated in the study up to approximately 4 weeks (28 days). Patients who remain hospitalized at the end of 28 days are followed through Day 42, death or discharge, whichever is sooner.

NasoVAX is an E1/E3-deleted, replication-deficient, adenovirus serotype 5 (RD-Ad5) vector that expresses the hemagglutinin (HA) surface antigen of an A/California/04/2009 (H1N1)-like influenza strain by way of a transgene insert (an investigational agent being studied under IND 21210; also known as AdcoCA09.HA). See Example 4a. The NasoVAX immunogenic composition (“drug product”) consists of a suspension of NasoVAX vector particles supplied as a frozen suspension in single-use dose vials containing 0.7 mL of drug product in a 2 mL USP Type 1 borosilicate glass vial. The frozen suspension is configured to deliver a nominal dose of 1×10¹¹ virus particles (vp). A 0.5 mL dose of the NasoVAX drug product (thawed to room temperature, about 25° C.) is administered to deliver 0.25 mL of an intranasal spray to each nostril with the subject in a supine position followed by a post-dose period of 30 minutes following administration in the supine position, preferably followed by observation for a sufficient period of time (e.g., two hours) after administration. The dose is designed to deliver about 1×10¹¹ vp and is administered using a one (1) mL Luer-lock syringe fitted with a Teleflex LMA MAD300 Nasal Intranasal Mucosal Atomization Device. Two 0.1-mL dose dividers are applied to the syringe plunger to enable a divided dose (i.e., one-half dose into each nostril, meaning that a sterile Teleflex LMA MAD atomizer is attached to the Luer-lock to deliver 0.25 mL of an intranasal spray to each nostril). As a placebo, commercially available 0.9% Sodium Chloride injectable solution for intranasal administration is prepared in syringes identical to those used for the NasoVAX dose and administered in the same manner as the NasoVAX dose.

Prior to discharge from the study center on the first day following administration of the drug product or placebo (“Day 1”), patients are provided with: a fingertip pulse oximetry device connecting to a tablet that will be supplied to the patient to measure resting SpO₂ and resting pulse rate remotely (patients are trained on the use of the device and tablet); a digital thermometer for measurement of oral temperature; and, instructions regarding how to take the above measurements and access and complete a web-based electronic diary (eDiary). After discharge on Day 1, patients return home for the duration of the study. Patients measure resting SpO₂, resting pulse rate and oral temperature twice daily at 09:00 hours and 16:00 hours, with a one (1)-hour window around each time point, and at any time they experience worsening symptoms of COVID-19 until Day 14 following administration of the drug product or placebo (“Day 14”) or hospitalization, whichever comes first. Patients are to record COVID-19-related symptoms daily during this same time period. Study center personnel contact patients daily by telephone during this period to document the patient's clinical status, record concomitant medications and monitor for AEs. After Day 14, study center personnel contact patients via telephone approximately every 7±2 days for 14 additional days to determine if they were hospitalized and to follow AEs that were not resolved by Day 14. If the patient is hospitalized at any time, the dates and times of hospital admission/discharge, ICU admission/discharge, and ventilation requirement are recorded, as applicable. Patients who remain hospitalized at the end of 28 days are followed through Day 42, death or discharge, whichever is sooner.

Several design features are employed in this study in an effort to minimize bias, including a double-blind design, with patients randomly assigned to receive NasoVAX or placebo on a 1:1 basis. Random assignment of patients avoids bias and helps ensure that both known and unknown risk factors are distributed evenly between treatment groups. The use of placebo control permits prospective comparison between the active treatment group and the control group. In order to mitigate the risk of exposure to SARS-CoV-2 at the study center and reduce the use of personal protective equipment, all data after administration of study drug is collected remotely, either electronically or via telephone; no in-person visits postvaccination are required unless a participant experiences a change in symptoms or AE that requires a visit for assessment. After study drug administration on Day 1, patients are evaluated remotely. The study design adheres to the March 2020 FDA Guidance on the conduct of clinical trials during the COVID-19 pandemic (United States Food and Drug Administration 2020).

The statistical methods used to study the data generated in this clinical trial include power and sample size assumptions considered sufficient by those of ordinary skill in the art. The sample size chosen for this study described above is considered adequate to meet the study objectives and was selected without statistical significance considerations. For the statistical analysis, the Safety Analysis Set is all patients who receive any NasoVAX; the Modified Intent To Treat (mITT) population are all randomized patients who receive any amount of NasoVAX and have a baseline and at least one post-baseline resting SpO₂ measurement (patients are analyzed according to the treatment that they receive); and the Per Protocol (PP) population are all randomized patients who receive any amount of NasoVAX according to the correct treatment assignment and who have 80% of twice daily results from resting SpO₂ measurements through Day 14 or hospitalization. Baseline is defined as data collected closest to randomization prior to any study drug dosing. For resting SpO₂ and resting pulse rate, Baseline is defined as the average of the two measurements at screening. All analyses and summary statistics are presented by treatment group (NasoVAX or placebo) across the cohorts, as well as by treatment group pooled across all cohorts. Descriptive statistics, including the numbers and percentages for categorical variables and the numbers, means, standard deviations, medians, minimums and maximums for continuous variables are provided by treatment across cohorts. Patients are randomized 1:1 to NasoVAX or placebo and proceed in Cohorts 1, 2 or 3 (described above). The cohorts will be enrolled in ascending order, with Cohort 1 preceding Cohort 2, and Cohort 2 preceding Cohort 3. Cohort 2 will be stratified by age groups of 35-64 years and 65 years and above, whereas Cohort 3 will be stratified by age groups (ages 35-64 years, ages 65 and above) and co-morbidity (one or more risk factors present, no risk factors present). Patients in Cohorts 2 and 3 will be stratified first, and then randomized 1:1 to NasoVAX or placebo. Enrollment of patients ages 35-49 is not to exceed approximately 30% of the randomized population of either treatment arm. In the efficacy analyses, descriptive statistics are used to evaluate differences between Baseline and post-Baseline efficacy endpoints. For the primary analysis, proportions of patients with “clinical worsening”, defined as a 4.0% decrease from Baseline in mean resting SpO2 on two consecutive measurements during the 14 days of home follow-up, or hospitalization, will be compared between NasoVAX and placebo groups using the Cochrane Mantel Haenszel test, while considering the stratification of age groups and risk factors, at a one-sided significance level of 0.025. The same approach is applied for secondary or exploratory endpoints that are categorical in nature. Sensitivity analyses is performed to assess the effect of site on the response to study drug. Linear and logistic regression are employed to examine the effects of Baseline factors, such as age, sex, medications, and medical co-morbidities on response. Changes from baseline in severity of COVID-19, is assessed by maximum decrease in resting SpO₂ or increase in resting pulse rate by outpatient pulse oximetry and COVID-19 symptoms (eDiary) during home follow-up, and analyzed using a mixed model for repeated measures. The model will include the fixed effects of treatment, week, and treatment-by-visit interaction as well as the continuous, covariate of Baseline level. The model employs an unstructured within patient covariance matrix and a restricted maximum likelihood estimation method. A Kaplan-Meier method is applied to compare changes between treatment groups in resting SpO₂ over time. For the purpose of study efficacy endpoints, resting SpO₂ and resting pulse rate are assessed on observed data only; if data is missing, it will not be imputed.

Replication deficient adenoviral vector with or without expressing an antigen transgene (e.g., NasoVAX) may be safe for administration to human beings having early COVID in the supine position and effective in preventing clinical worsening in such patients. In certain embodiments, any replication deficient adenovirus vector (with or without expressing an antigen transgene) administered intranasally may be used in the present methods for prevention of worsening COVID symptoms during early infection. In certain embodiments, intranasal administration of the present replication deficient adenoviral vector, with or without expressing an antigen transgene, rapidly (e.g., within 24 hours, or 1-2 days post administration) induces a pan-specific response to a viral pathogen to permit treatment before the pathogen is identified or understood. In certain other embodiments, intranasal administration of the present replication deficient adenoviral vector, with or without expressing an antigen transgene, is used concomitantly with antivirals or prophylactic vaccines. The protective effects of NasoVAX may be viewed as a biologic response modulating innate immunity that dampens the excessive and pathogenic immune response to a respiratory pathogen.

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A method for transmucosal administration of a therapeutic dose of an immunogenic composition comprising an antigen from a pathogen to a mammalian subject, wherein the method comprises:
administering intranasally to the mammalian subject an effective amount of the immunogenic composition,
wherein the subject during administration is in a modified sitting, reclining, supine, or other dosing position wherein nostrils of the mammalian subject are tilted upward;
whereby the therapeutic dose administered transmucosally induces a protective immune response;
optionally wherein the immunogenic composition is a vaccine.

2. The method of claim 1, further comprising instructing or otherwise keeping the mammalian subject to remain in the dosing position during a post-dose hold period selected from about 5 minutes to about 30 minutes.

3. A method for vaccinating a human against viral infection, comprising:
intranasally administrating an effective amount of a therapeutic dose of an immunogenic composition to the human, wherein the immunogenic composition comprises an antigen from a pathogen to the human, wherein during the intranasal administration the human is in a modified sitting, reclining, or supine position, wherein nostrils of the human are tilted upward; and
instructing or otherwise keeping the human to remain in the modified sitting, reclining, or supine position for about 5 minutes, 10 minutes, 15 minutes, 20 minutes, 25 minutes, or 30 minutes after the intranasal administration,
whereby the therapeutic dose administered intranasally induces a protective immune response in the human.

4. A method for treating viral infection in a human, comprising:
intranasally administrating an effective amount of a therapeutic dose of an immunogenic composition to the human, wherein the immunogenic composition comprises an antigen from a pathogen to the human, wherein during the intranasal administration the human is in a modified sitting, reclining, or supine position, wherein nostrils of the human are tilted upward; and
instructing or otherwise keeping the human to remain in the modified sitting, reclining, or supine position for about 5 minutes, 10 minutes, 15 minutes, 20 minutes, 25 minutes, or 30 minutes after the intranasal administration,
whereby the therapeutic dose administered intranasally induces a protective immune response in the human.

5. The method of any preceding claim, wherein the nostrils of the mammalian subject or human are elevated above a head of the mammalian subject or human.

6. The method of any preceding claim, wherein less than about 20% of the therapeutic dose leaves the nasal cavity via drainage externally.

7. The method of any preceding claim, wherein the immunogenic composition is administered as a single dose.

8. The method of any one of claims 1-6, wherein the immunogenic composition is administered as a divided dose in each nostril.

9. The method of claim 8, wherein the divided dose is administered using a multidose intranasal delivery device.

10. The method of any preceding claim, wherein the immunogenic composition is provided as a prime dose and a boost dose.

11. The method of any preceding claim, wherein the immunogenic composition is provided as a dosing regimen in combination with a heterologous dose as a prime dose or boost dose.

12. The method of any preceding claim, wherein the immunogenic composition comprises a non-replicating viral vector that encodes and expresses a heterologous antigen.

13. The method of any one of claims 1-11, wherein the immunogenic composition comprises a non-replicating viral vector without encoding a heterologous antigen.

14. The method of any preceding claim, wherein the immunogenic composition comprises a non-replicating adenovirus vector.

15. The method of claim 14, wherein the adenovirus is a human adenovirus.

16. The method of claim 14, wherein the adenovirus is selected from the group consisting of a bovine adenovirus, a canine adenovirus, a non-human primate adenovirus, a chicken adenovirus, or a porcine and a swine adenovirus.

17. The method of any one of claims 14-16, wherein the adenovirus vector is an E1 and E3 deleted adenovirus vector.

18. The method of any preceding claim, wherein the effective amount is selected from the group consisting of at least 10⁷ viral particle (vp) of E1 and/or E3 deleted or disrupted adenovirus vector, at least 10⁸ viral particle (vp) of E1 and/or E3 deleted or disrupted adenovirus vector, at least 10⁹ viral particle (vp) of E1 and/or E3 deleted or disrupted adenovirus vector, at least 10¹⁰ viral particle (vp) of E1 and/or E3 deleted or disrupted adenovirus vector, and at least 10¹¹ viral particle (vp) of E1 and/or E3 deleted or disrupted adenovirus vector.

19. The method of any preceding claim, wherein the mammalian subject or human is administered intranasally the immunogenic composition comprising an effective amount of at least 10⁷ viral particle (vp) of replication deficient adenovirus vector that contains and expresses a heterologous antigen codon optimized for the mammalian subject or human.

20. The method of any preceding claim, wherein the pathogen is a respiratory pathogen.

21. The method of any preceding claim, wherein the pathogen is a virus.

22. The method of claim 21, wherein the virus is selected from the group consisting of an orthomyxovirus, a paramyxovirus, a rhinovirus and a coronavirus.

23. The method of claim 22, wherein the coronavirus is selected from the group consisting of SARS-Cov, SARS-Cov-2, MERS, human coronaviruses 229E (HCoV-229E), OC43 (HCoV-OC43), NL63, and HKU1.

24. The method of claim 21, wherein the virus is selected from the group consisting of an influenza virus, a respiratory syncytial virus (RSV), a common cold virus and a measles virus.

25. The method of claim 24, wherein the common cold virus is a rhinovirus or a coronavirus.

26. The method of any one of claims 1-20, wherein the pathogen is a bacterium.

27. The method of claim 26, wherein the bacterium is selected from the group consisting of Bacillus, Mycobacterium, Staphylococcus, Streptococcus, Pseudomonas, Klebsiella, Haemophilus, and Mycoplasma.

28. The method of claim 27, wherein the bacterium is Bacillus anthracis.

29. The method of claim 27 or 28, wherein the antigen is Bacillus anthracis protective antigen.

30. The method of claim 29, wherein the Bacillus anthracis protective antigen is PA83.

31. The method of claim 27 or 28, wherein the antigen is Bacillus anthracis lethal factor.

32. The method of any one of claims 1-20, wherein the pathogen is a fungus.

33. The method of claim 32, wherein the fungus is Aspergillus.

34. The method of any one of claims 1-22, wherein the antigen is an influenza antigen selected from the group consisting of a seasonal influenza virus antigen, an influenza A virus antigen, an influenza B virus antigen.

35. The method of any one of claims 1-22 and 34, wherein the antigen is a hemagglutinin (HA) surface protein influenza antigen, or fragment thereof.

36. The method of claim 34 or 35, wherein the therapeutic dose of the immunogenic composition comprises a monovalent influenza pharmaceutical formulation suitable for a single dose intranasal administration to a human subject, the immunogenic composition comprising:
an effective amount of at least 10⁸ viral particles (vp) of replication deficient adenovirus vector that contains and expresses influenza virus hemagglutinin antigen codon optimized for the human subject, wherein the effective amount induces a protective immune response configured to provide seroprotection to the human subject of a hemagglutination inhibition assay (HAI) antibody titer≥40 within 28 days after administration against the influenza virus; and,
a pharmaceutically acceptable diluent or carrier.

37. The method of claim 36, wherein:
a) the formulation is configured to provide seroprotection to the human subject of an HAI antibody titer≥40 within 28 days post administration against the influenza virus, optionally wherein the HAI antibody titer is at least 50 and/or within 28 days post administration;
b) the effective amount is at least 10⁹ or at least 10¹⁰ viral particles (vp);
c) the formulation does not comprise an adjuvant;
d) the influenza virus HA antigen is from an influenza A virus, optionally wherein the influenza A virus is subtype H1N1 and/or subtype H3N2;
e) the method induces a seroprotective immune response lasting for at least 12 months;
f) the method induces a combined mucosal, humoral and T cell protective immune response and provides protection against influenza A virus and influenza B virus subtype; and/or,
g) the immune response is measured by the haemagglutination inhibition assay (HAI).

38. The method of any one of claims 1-2 and 5-35, wherein the mammalian subject is a companion animal, a domesticated animal, a food- or feed-producing animal, a livestock animal, a game animal, a racing animal, or a sport animal.

39. The method of any one of claims 1-2 and 5-35, wherein the mammalian subject is a cow, a horse, a rabbit, a dog, a cat, a goat, a sheep, or a pig.

40. The method of any one of claims 1-2 and 5-35, wherein the mammalian subject is a human being or human subject.

41. A method for transmucosal administration of a therapeutic dose of a non-replicating viral vectored immunogenic composition to a mammalian subject, wherein the method comprises:
administering intranasally to the mammalian subject an immunogenic composition comprising an effective amount of at least 10⁸ viral particle (vp) of replication deficient adenovirus vector, with or without containing and expressing a heterologous antigen codon optimized for the mammalian subject,
wherein the subject during administration is in a modified sitting, reclining, supine, or other dosing position wherein nostrils of the mammalian subject are tilted upward;
whereby the therapeutic dose administered transmucosally induces a protective immune response;
optionally wherein the immunogenic composition is a vaccine.

42. The method of claim 41, further comprising instructing or otherwise keeping the mammalian subject to remain in the dosing position during a post-dose hold period selected from about 5 minutes to about 30 minutes

43. A method for vaccinating a human against viral infection, comprising:
intranasally administrating an effective amount of a therapeutic dose of an immunogenic composition to the human, wherein the immunogenic composition comprises an effective amount of at least 10⁸ viral particle (vp) of replication deficient adenovirus vector, with or without encoding a heterologous antigen from a pathogen to the human, wherein during the intranasal administration the human is in a modified sitting, reclining, or supine position, wherein nostrils of the human are tilted upward; and
instructing or otherwise keeping the human to remain in the modified sitting, reclining, or supine position for about 5 minutes, 10 minutes, 15 minutes, 20 minutes, 25 minutes, or 30 minutes after the intranasal administration,
whereby the therapeutic dose administered intranasally induces a protective immune response in the human.

44. A method for treating viral infection in a human, comprising:
intranasally administrating an effective amount of a therapeutic dose of an immunogenic composition to the human, wherein the immunogenic composition comprises an effective amount of at least 10⁸ viral particle (vp) of replication deficient adenovirus vector, with or without encoding a heterologous antigen from a pathogen to the human, wherein during the intranasal administration the human is in a modified sitting, reclining, or supine position, wherein nostrils of the human are tilted upward; and
instructing or otherwise keeping the human to remain in the modified sitting, reclining, or supine position for about 5 minutes, 10 minutes, 15 minutes, 20 minutes, 25 minutes, or 30 minutes after the intranasal administration,
whereby the therapeutic dose administered intranasally induces a protective immune response in the human.

45. The method of any one of claims 41-44, wherein the nostrils of the mammalian subject or human are elevated above a head of the mammalian subject or human.

46. The method of any one of claims 41-45, wherein less than about 20% of the pharmaceutical composition leaves the nasal cavity via drainage externally.

47. The method of any one of claims 41-46, wherein the immunogenic composition is administered as a single dose.

48. The method of any one of claims 41-46, wherein the immunogenic composition is administered as a divided dose in each nostril.

49. The method of claim 48, wherein the divided dose is administered using a multidose intranasal delivery device.

50. The method of any one of claims 41-49, wherein the immunogenic composition is provided as a prime dose and a boost dose.

51. The method of any one of claims 41-49, wherein the immunogenic composition is provided as a dosing regimen in combination with a heterologous dose as a prime dose or boost dose.

52. The method of any one of claims 41-51, wherein the protective immune response is induced within about 24 hours.

53. The method of any one of claims 41-51, wherein the protective immune response is induced within about 1 or 2 days.

54. The method of any one of claims 41-53, wherein the adenovirus is a human adenovirus.

55. The method of any one of claims 41-53, wherein the adenovirus is selected from the group consisting of a bovine adenovirus, a canine adenovirus, a non-human primate adenovirus, a chicken adenovirus, or a porcine and a swine adenovirus.

56. The method of any one of claims 41-55, wherein the adenovirus vector is an E1 and E3 deleted adenovirus vector.

57. The method of any one of claims 41-55, wherein the effective amount is selected from the group consisting of at least 10⁹ viral particle (vp) of E1 and/or E3 deleted or disrupted adenovirus vector, at least 10¹⁰ viral particle (vp) of E1 and/or E3 deleted or disrupted adenovirus vector, and at least 10¹¹ viral particle (vp) of E1 and/or E3 deleted or disrupted adenovirus vector.

58. The method of any one of claims 41-57, wherein the heterologous antigen is from a respiratory pathogen.

59. The method of claim 58, wherein the respiratory pathogen is a virus.

60. The method of claim 59, wherein the virus is selected from the group consisting of an orthomyxovirus, a paramyxovirus, a rhinovirus and a coronavirus.

61. The method of claim 60, wherein the coronavirus is selected from the group consisting of SARS-Cov, SARS-Cov-2, MERS, human coronavirus 229E (HCoV-229E), OC43 (HCoV-OC43), NL63, and HKU1.

62. The method of claim 59, wherein the virus is selected from the group consisting of an influenza virus, a respiratory syncytial virus (RSV), a common cold virus and a measles virus.

63. The method of claim 62, wherein the common cold virus is a rhinovirus or a coronavirus.

64. The method of claim 58, wherein the respiratory pathogen is a bacterium selected from the group consisting of Bacillus, Mycobacterium, Staphylococcus, Streptococcus, Pseudomonas, Klebsiella, Haemophilus, and Mycoplasma.

65. The method of claim 58, wherein the respiratory pathogen is a fungus that is optionally Aspergillus.

66. The method of any one of claims 41-58 and 64, wherein the heterologous antigen is Bacillus anthracis protective antigen, optionally PA83 and/or Bacillus anthracis lethal factor.

67. The method of any one of claims 41-60 and 62, wherein the heterologous antigen is an influenza antigen, optionally a hemaglutinnin (HA) surface protein antigen, and:
a) the composition is configured to provide seroprotection to a mammalian subject of an HA1 antibody titer≥40 within 28 days post administration against the influenza virus, optionally wherein the HA1 antibody titer is at least 50 and/or within 28 days post administration;
b) the effective amount is at least 10⁹ or at least 10¹⁰ viral particles (vp);
c) the composition does not comprise an adjuvant;
d) the influenza virus HA antigen is from an influenza A virus, optionally wherein the influenza A virus is subtype H1N1 and/or subtype H3N2;
e) the method induces a seroprotective immune response lasting for at least 12 months; and/or,
f) the method induces a combined mucosal, humoral and T cell protective immune response provides protection against influenza A virus and influenza B virus subtype.

68. The method of any one of claims 41-42 and 45-67, wherein the mammalian subject is a companion animal, a domesticated animal, a food- or feed-producing animal, a livestock animal, a game animal, a racing animal, or a sport animal.

69. The method of any one of claims 41-42 and 45-68, wherein the mammalian subject is a cow, a horse, a rabbit, a dog, a cat, a goat, a sheep, or a pig.

70. The method of any one of claims 41-42 and 45-67, wherein the mammalian subject or patient is a human.

71. The method of any one of claims 41-67 and 70, the method comprising intranasally administering into the nostrils of a human subject the effective amount of the immunogenic composition, wherein: during administration the nostrils of the human subject are tilted upward; and, the effective amount induces a protective immune response against a respiratory pathogen.

72. The method of claim 71, wherein the effective amount comprises at least about 10⁹ vp.

73. The method of claim 71 or 72, wherein immunogenic composition is administered in two doses split approximately evenly between two nostrils of the human subject.

74. The method of any one of claims 71-73, wherein the human subject is in the supine position during administration and remains in the supine position for at least about 30 minutes following administration.

75. The method of any one of claims 71-74, wherein the heterologous antigen is an influenza antigen, optionally wherein the influenza antigen is the hemagglutinin (HA) surface antigen.

76. The method of any one of claims 71-74, wherein the heterologous antigen is a coronavirus antigen, optionally wherein the coronavirus antigen is a SARS-Cov-2 antigen.

77. The method of any one of claims 71-74, wherein the replication deficient adenovirus vector does not contain or express a heterologous antigen.

78. The method of any one of claims 71-77, wherein the human subject is a human being with early coronavirus infectious disease.

79. The method of claim 78, wherein the method prevents clinical worsening in the human being.

80. The method of claim 79, wherein the clinical worsening is at least about a 4.0% decrease from baseline in resting SpO₂ as determined by pulse oximetry on two consecutive measurements following administration.

81. The method of claim 79 or 80 wherein the clinical worsening is determined by measuring at least one characteristic selected from the group consisting of maximal severity of COVID-19 after treatment as assessed by average decrease in resting SpO₂ from baseline resting SpO₂ at screening during the 14 days following administration, average increase in resting pulse rate from baseline resting pulse rate at screening during the 14 days following administration, proportion of patients requiring hospitalization on Day 42 following administration according to the level of oxygen supplementation required, and all-cause mortality through Day 42 following administration.

82. The method of any one of claims 71-81, wherein the protective immune response against a respiratory pathogen is elicited within about 24 hours.

83. The method of any one of claims 71-81, wherein the protective immune response against a respiratory pathogen is elicited within about 1 to 2 days.

84. The method of any one of claims 1-83, wherein the method comprises instructing or otherwise keeping the mammalian subject or human to remain in the modified sitting, reclining, or supine position for 30 minutes after the intranasal administration.

85. A method for transmucosal administration of a therapeutic dose of a non-replicating viral vectored immunogenic composition to a human subject, wherein the method comprises:
administering intranasally to the human subject an immunogenic composition comprising an effective amount of at least 10⁸ viral particle (vp) of replication deficient adenovirus vector, with or without containing and expressing a heterologous antigen codon optimized coding sequence for the human subject,
wherein the human subject during administration is in a modified sitting, reclining, supine, or other position wherein nostrils of the human subject are tilted upward;
whereby the therapeutic dose administered transmucosally induces a protective immune response against a respiratory pathogen.

86. The method of claim 85, wherein the respiratory pathogen is selected from the group consisting of a orthomyxovirus; a paramyxovirus; a rhinovirus; a coronavirus; an influenza virus, a respiratory syncytial virus (RSV); a common cold virus; and a measles virus.

87. The method of claim 85 or 86, wherein the replication deficient adenovirus vector contains and expresses a heterologous antigen selected from an influenza virus antigen or a coronavirus antigen.

88. The method of any one of claims 85-87, wherein the heterologous antigen is a SARS-Cov-2 antigen.

89. The method of any one of claims 85-87, wherein the heterologous antigen is an influenza antigen.
